SEQUENCE LISTING (73 sequences)

SEQ ID NO: 1 - DNA, 20 bp, Artificial Sequence
  Synthetic Primer
agtaactgggatgacatgga

SEQ ID NO: 2 - DNA, 20 bp, Artificial Sequence
  Synthetic Primer
taaccctcatagattggcac

SEQ ID NO: 3 - DNA, 21 bp, Artificial Sequence
  Synthetic Primer
tgacaatgggacaggaatggt

SEQ ID NO: 4 - DNA, 20 bp, Artificial Sequence
  Synthetic Primer
cagcccttggagcatcatct

SEQ ID NO: 5 - DNA, 24 bp, Artificial Sequence
  Synthetic Primer
tcacccacatccaaccgtccatct

SEQ ID NO: 6 - DNA, 24 bp, Artificial Sequence
  Synthetic Primer
tgcttaatttgtagctcccacaca

SEQ ID NO: 7 - DNA, 22 bp, Artificial Sequence
  Synthetic Primer
acaaagtaacgtcacaccaact

SEQ ID NO: 8 - DNA, 25 bp, Artificial Sequence
  Synthetic Primer
acaatgcatggggtctattcataac

SEQ ID NO: 9 - DNA, 24 bp, Artificial Sequence
  Synthetic Primer
tgccatattgctcaatgtgtcact

SEQ ID NO: 10 - DNA, 25 bp, Artificial Sequence
  Synthetic Primer
tcttaaaacccctctataagagtac

SEQ ID NO: 11 - DNA, 23 bp, Artificial Sequence
  Synthetic Primer
cgctagcatgcctgccgctaccg

SEQ ID NO: 12 - DNA, 31 bp, Artificial Sequence
  Synthetic Primer
cggatccttaggatttgactgctagagaatc

SEQ ID NO: 13 - DNA, 30 bp, Artificial Sequence
  Synthetic Primer
cgctagcatgcctgcctatgataacacttc

SEQ ID NO: 14 - DNA, 30 bp, Artificial Sequence
  Synthetic Primer
gctcgagttaagcagagtcttcttgcatgg

SEQ ID NO: 15 - DNA, 24 bp, Artificial Sequence
  Synthetic Primer
agaaggtccagctcttccagttct

SEQ ID NO: 16 - DNA, 47 bp, Artificial Sequence
  Synthetic Primer
gcgtaatacgactcactatagggagaaggtccagctcttccagttct

SEQ ID NO: 17 - DNA, 25 bp, Artificial Sequence
  Synthetic Primer
actaaccaatgcaattggcttcaaa

SEQ ID NO: 18 - DNA, 48 bp, Artificial Sequence
  Synthetic Primer
gcgtaatacgactcactatagggactaaccaatgcaattggcttcaaa

SEQ ID NO: 19 - DNA, 24 bp, Artificial Sequence
  Synthetic Primer
gggattggaatttacaccagtgag

SEQ ID NO: 20 - DNA, 47 bp, Artificial Sequence
  Synthetic Primer
gcgtaatacgactcactataggggggattggaatttacaccagtgag

SEQ ID NO: 21 - DNA, 27 bp, Artificial Sequence
  Synthetic Primer
ggcaaataattgagttgaatgctcaag

SEQ ID NO: 22 - DNA, 50 bp, Artificial Sequence
  Synthetic Primer
gcgtaatacgactcactatagggggcaaataattgagttgaatgctcaag

SEQ ID NO: 23 - DNA, 20 bp, Artificial Sequence
  Synthetic Primer
atcaaaagctgaaagacttg

SEQ ID NO: 24 - DNA, 43 bp, Artificial Sequence
  Synthetic Primer
gcgtaatacgactcactatagggatcaaaagctgaaagacttg

SEQ ID NO: 25 - DNA, 23 bp, Artificial Sequence
  Synthetic Primer
ttggtacaatctttaaaactagt

SEQ ID NO: 26 - DNA, 46 bp, Artificial Sequence
  Synthetic Primer
gcgtaatacgactcactatagggttggtacaatctttaaaactagt

SEQ ID NO: 27 - DNA, 25 bp, Artificial Sequence
  Synthetic Primer
gatcttgttaaagtgggaggtgtga

SEQ ID NO: 28 - DNA, 21 bp, Artificial Sequence
  Synthetic Primer
gtgcaaccacagatccattcc

SEQ ID NO: 29 - DNA, 22 bp, Artificial Sequence
  Synthetic Primer
agggatgttgcattagctcaca

SEQ ID NO: 30 - DNA, 27 bp, Artificial Sequence
  Synthetic Primer
tcagcacataagtatctaccagaagca

SEQ ID NO: 31 - DNA, 19 bp, Artificial Sequence
  Synthetic Primer
gcgaccaaaccgtgtgtgt

SEQ ID NO: 32 - DNA, 25 bp, Artificial Sequence
  Synthetic Primer
aagagaagtttgacaagccaagaag

SEQ ID NO: 33 - DNA, 22 bp, Artificial Sequence
  Synthetic Primer
aaagtgattgtggcagaatgca

SEQ ID NO: 34 - DNA, 19 bp, Artificial Sequence
  Synthetic Primer
ttttgtggccaggcttgaa

SEQ ID NO: 35 - DNA, 30 bp, Artificial Sequence
  Synthetic Primer
tccttgttggattggtagccaactttgttg

SEQ ID NO: 36 - DNA, 1011 bp, Medicago truncatula
atgcctgcctatgataacacttcatcagtctccggtggcgaccaaaccgtgtgtgtcaccggtgctggcgggtttatagcttcttggcttgtcaaacttctcttggaaagaggttacactgttagaggaaccgtccgaaacccagaggatccaaagaatggtcacttaaaagaattggaaggagcaagagagaggttaactttgcataaggttgatcttcttgatcttcaatctatccaatctgttgttcatggttgccatggtgtgttccacacagcctctcctgtcactgacaaccctgatgagatgttagagccagcagtgaatggaactaagaatgtgattatagcgtccgcggaagctaaagtgcgacgtgttgttttcacttcatcgattggaacggtctatatggacccaaatactagtagagatgttgtcgttgatgaatcatattggagtgatttagagcattgcaagaacaccaagaactggtattgttatgggaagacagtggctgaacaatcagcatgggatatagcaaaagaaaatcaagttgatttggttgtggtgaacccagttgtagttcttggaccattgttacaaccaacaattaatgctagtacaattcacatactcaagtaccttaatggtgctgcaaaaacttatgtgaatgcaacacaatcttatgttcatgttaaggatgtggcattagcacatcttcttgtttatgagacaaattctgcttctggtagatacatatgttgtgaaactgctttgcatcgtggtgaggttgttgaaattttggccaagtattttcctgagtacccacttcctaccaaatgttcagatgaaaagaatccaagagtgaaaccttataaattttcaaatcaaaagctgaaagacttgggattggaattcacacctgtgaagcaatgtttatatgacactgttaggagtctacaagagaaaggacaccttcctattcctcccatgcaagaagactctgcttaa

SEQ ID NO: 37 - protein, 336 aa, Medicago truncatula
MPAYDNTSSVSGGDQTVCVTGAGGFIASWLVKLLLERGYTVRGTVRNPEDPKNGHLKELEGARERLTLHKVDLLDLQSIQSVVHGCHGVFHTASPVTDNPDEMLEPAVNGTKNVIIASAEAKVRRVVFTSSIGTVYMDPNTSRDVVVDESYWSDLEHCKNTKNWYCYGKTVAEQSAWDIAKENQVDLVVVNPVVVLGPLLQPTINASTIHILKYLNGAAKTYVNATQSYVHVKDVALAHLLVYETNSASGRYICCETALHRGEVVEILAKYFPEYPLPTKCSDEKNPRVKPYKFSNQKLKDLGLEFTPVKQCLYDTVRSLQEKGHLPIPPMQEDSA

SEQ ID NO: 38 - DNA, 1196 bp, Populus trichocarpa
atgcctgtcgatacttcatcacttccatgccaaggccaaactgtctgtgtcaccggggctggtggcttcattgcttcatggattgttaaacttcttctagagaaaggttactctgttaaaggaactgtgaggaacccagctgatcccaagaattcccatttgagggagcttgaaggagctcaagaaagattaactttatgcaaggctgatattcttgattatgagtctcttaaagaggctattcaagggtgtgatggagttttccatactgcttgtcctgtcacagacgatccagacaaggtgatggagccagcagtgaatggaaccaagaatgtgatcatggcagcagctgaggccaaagtccgacgagtggttttcacgtcctcaattggtactgtgtacatggaccccaataggagccctgatgttgtcgttgatgaatcttgctggagtgatttgagtattgcaagaacaccaagaattggtattgctatggaaagactgtggcagaacaggttgcatgggatgtggctaagaagaaaggagttgacctagtggtggtgaacccagtggtggtgcttggaccattgttgcaacccactgtcaatgctagcatccttcacatcctcaagtacctaaccggctcagccaagacatatgctaacgctgttcaagcttatgtgcatgttagggatgtggccgtagcccacattttagtcttcgagacaccttctgcctccggccgttacatttgctttgagaaaatgcttcaccgtggagaggtggtggaaatccttgcaaagttcttcccggagtatcccatccccaccaagtgttctgatgagaagaacccaagaaaacaaaactacaagctcacaaaccagaagatcaaggatctgggcatcgaattcgtcccagtaaaacagtgcttgtatgaaactgttaagagcttgcaggaaaagggtatccttccaatcctaaaacatgctgaagactctgtgaaaattcaataaggctcattgattagcagggtataggtccacacctcaagtatggttgaagttgtttgtaaaatacgtactaaaaaaaagaatatactaaagaagaatgtcaatgtcagagtgtaagtgttactgtcttctatatgtaaagacttctaattgtcttcgtgcttagattaatccttttataatgaa

SEQ ID NO: 39 - protein, 336 aa, Populus trichocarpa
MPVDTSSLPCQGQTVCVTGAGGFIASWIVKLLLEKGYSVKGTVRNPADPKNSHLRELEGAQERLTLCKADILDYESLKEAIQGCDGVFHTACPVTDDPDKVMEPAVNGTKNVIMAAAEAKVRRVVFTSSIGTVYMDPNRSPDVVVDESCWSDLEYCKNTKNWYCYGKTVAEQVAWDVAKKKGVDLVVVNPVVVLGPLLQPTVNASILHILKYLTGSAKTYANAVQAYVHVDVAVAHILVFETPSASGRYICFEKMLHRGEVVEILAKFFPEYPIPTKCSDEKNPRKQNYKLTNQKIKDLGIEFVPVKQCLYETVKSLQEKGILPILKHAEDSVKIQ

SEQ ID NO: 40 - DNA, 1198 bp, Populus trichocarpa
atgcctgtcgatacttcatcacttccatgccaaggccaaactgtctgtgtcaccggggctggtggcttcattgcttcatggattgttaaacttcttctagagaaaggttactctgttaaaggaactgtgaggaacccagctgatcccaagaattcccatttgagggagcttgaaggagctcaagaaagattaactttatgcaaggctgatattcttgattatgagtctcttaaagaggctattcaagggtgtgatggagttttccatactgcttgtcctgtcacagacgatccagacaaggtgatggagccagcagtgaatggaaccaagaatgtgatcatggcagcagctgaggccaaagtccgacgagtggttttcacgtcctcaattggtactgtgtacatggaccccaataggagccctgatgttgtcgttgatgaatcttgctggagtgatcttgagtattgcaagaacaccaagaattggtattgctatggaaagactgtggcagaacaggttgcatgggatgtggctaagaagaaaggagttgacctagtggtggtgaacccagtggtggtgcttggaccattgttgcaacccactgtcaatgctagcatccttcacatcctcaagtacctaaccggctcagccaagacatatgctaacgctgttcaagcttatgtgcatgttagggatgtggccgtagcccacattttagtcttcgagacaccttctgcctccggccgttacatttgctttgagaaaatgcttcaccgtggagaggtggtggaaatccttgcaaagttcttcccggagtatcccatccccaccaagtgttctgatgagaagaacccaagaaaacaaaactacaagctcacaaaccagaagatcaaggatctgggcatcgaattcgtcccagtaaaacagtgcttgtatgaaactgttaagagcttgcaggaaaagggtatccttccaatcctaaaacatgctgaagactctgtgaaaattcaataagggctcattgattagcagggtataggtccacacctcaagtatggttgaagttgtttgtaaaatacgtactaaaaaaaagaatatactaaagaagaatgtcaatgtcagagtgtaagtgttactgtcttctatatgtaaagacttctaattgtcttcgtgcttagattaatccttttataatgaa

SEQ ID NO: 41 - protein, 337 aa, Populus trichocarpa
MPVDTSSLPCQGQTVCVTGAGGFIASWIVKLLLEKGYSVKGTVRNPADPKNSHLRELEGAQERLTLCKADILDYESLKEAIQGCDGVFHTACPVTDDPDKVMEPAVNGTKNVIMAAAEAKVRRVVFTSSIGTVYMDPNRSPDVVVDESCWSDLEYCKNTKNWYCYGKTVAEQVAWDVAKKKGVDLVVVNPVVVLGPLLQPTVNASILHILKYLTGSAKTYANAVQAYVHVRDVAVAHILVFETPSASGRYICFEKMLHRGEVVEILAKFFPEYPIPTKCSDEKNPRKQNYKLTNQKIKDLGIEFVPVKQCLYETVKSLQEKGILPILKHAEDSVKIQ

SEQ ID NO: 42 - DNA, 1026 bp, Populus trichocarpa
atgcctgtcgatacttcatcacttccatgccaaggccaaactgtctgtgtcaccggggctggtggcttcattgcttcatggattgttaaacttcttctagagaaaggttactctgttaaaggaactgtgaggaacccagctgatcccaagaattcccatttgagggagcttgaaggagctcaagaaagattaactttatgcaaggctgatctccttgattatgagtctcttaaagaggctattcaagggtgtgatggagttttccatactgcttctcctctcacagacgatccagagcaaatggtggagccagcagtgaatggatccaagaatgtgatcatggcagcatctgaggccaaagtccgacgagtggttttcacgtcttcaattggtactgtgtacatggaccccaataggagccctgatgttgtcgttgatgaatcttgctggagtgatcttgagtattgcaagaacaccaagaattggtattgctatggaaagactgtggcagaacaggttgcatgggatgtggctaagaagaaaggagttgacctagtggtggtgaacccagtggtggtgcttggaccattgttgcaacccactgtcaatgctagcatccttcacatcctcaagtacctaaccggctcagccaagacatatgctaacgctgttcaagcttatgtgcatgttagggatgtggcggtagcccacattttagtcttcgagacaccttctgcctccggccgttacatttgctttgagaaaatgcttcaccgtggagaggttgtggaaatccttgcaaagttcttcccggagtatcccatccccaccaagtgttcagatgagaagaacccaagaaaacaaccctacaagttcacaaaccagaagatcaaggatctgggcatcgaattcaccccagtgaaacagtgtctgtatgaaactgttaagagcttgcaggaaaagggtcaccttccaatcccaaaacaagctaaagatgggtccgttgtcagaatctccaaatattaa

SEQ ID NO: 43 - protein, 341 aa, Populus trichocarpa
MPVDTSSLPCQGQTVCVTGAGGFIASWIVKLLLEKGYSVKGTVRNPADPKNSHLRELEGAQERLTLCKADLLDYESLKEAIQGCDGVFHTASPLTDDPEQMVEPAVNGSKNVIMAASEAKVRRVVFTSSIGTVYMDPNRSPDVVVDESCWSDLEYCKNTKNWYCYGKTVAEQVAWDVAKKKGVDLVVVNPVVVLGPLLQPTVNASILHILKYLTGSAKTYANAVQAYVHVRDVAVAHILVFETPSASGRYICFEKMLHRGEVVEILAKFFPEYPIPTKCSDEKNPRKQPYKFTNQKIKDLGIEFTPVKQCLYETVKSLQEKGHLPIPKQAKDGSVVRISKY

SEQ ID NO: 44 - DNA, 1031 bp, Populus trichocarpa
atgcctgtcgatacttcatcacttccatgccaaggccaaactgtctgtgtcaccggggctggtggcttcattgcttcatggattgttaaacttcttctagagaaaggttactctgttaaaggaactgtgagaaacccagctgatcccaagaattcccatttgagggagcttgaaggagctcaagaaagattaactttatgcaaggctgatctccttgattatgagtctcttaaagaggctattcaagggtgtgatggagttttccatactgcttctcctgtcacagacgatccagagcaaatgctggagccagcagtgaatggaaccaagaatgtgatcatggcagcagctgaggccaaagtccgacgagtggttttcacgtcttcaattgggactgtgtacatggaccccaataggagccctgatgttgtcgttgatgaatcttgctggagtgatcttgagttctgcaagaacaccaagaattggtattgctatggaaagactgtggcggaacaggatgcatgggatgtggctaagaagaatggagttgacctagtggtggtgaacccagtgctggtgcttggaccattgttgcaacccactgtcaatgctagcatcgttcacatcctcaagtacctaaccggctcagccaagacatatgctaactctgttcaagcttatgtgcatgttaaggatgtggcactagcccacattttagtcttcgagacaccttccgcctccggccgttacatttgcgctgagagaatgctccaccgtggagaggtggtggaaatccttgcaaagttcttcccggagtatcccatccccaccaagtgttcagatgagaagaatccaagaaaacaaccctacaagttcacaaaccagaagatcaaggatctgggcatcgaattcaccccagtgaaacagtgcctgtatgaatctgttaagagcttgcaggaaaagggtcaccttccaatcccaaaacaagctgaagactctgtgaaaattcaacaagggctcatttattagga

SEQ ID NO: 45 - protein, 342 aa, Populus trichocarpa
MPVDTSSLPCQGQTVCVTGAGGFIASWIVKLLLEKGYSVKGTVRNPADPKNSHLRELEGAQERLTLCKADLLDYESLKEAIQGCDGVFHTASPVTDDPEQMLEPAVNGTKNVIMAAAEAKVRRVVFTSSIGTVYMDPNRSPDVVVDESCWSDLEFCKNTKNWYCYGKTVAEQDAWDVAKKNGVDLVVVNPVLVLGPLLQPTVNASIVHILKYLTGSAKTYANSVQAYVHVKDVALAHILVFETPSASGRYICAERMLHRGEVVEILAKFFPEYPIPTKCSDEKNPRKQPYKFTNQKIKDLGIEFTPVKQCLYESVKSLQEKGHLPIPKQAEDSVKIQQGLIY

SEQ ID NO: 46 - DNA, 999 bp, Lycopersicon esculentum
atgccatcagaatccggcaaagttgtttgtgtcaccggcgccggcggtttcattgcctcttggctcgttaaactccttctagaaaaaggctacactgtcagaggaaccgttagaaaccctgatgattccaaaaatggtcacctgaaggaacttgaaggtgcaaaggagagactgattctgttgagagctgatcttctagactatcagagtttgagagaagcaatttatggctgtgacggagttttccacaccgcctcccctgtcactgatgatccagaacaaatggtggagccagcagttattgggaccaagaatgtgataacagcagcagcagaaaccaaagttcgcagagttgtgtttacttcttcaattggtacagtatacatggaccccaaccgggcccctgataaagttgtggacgagacttgctggagtgatcttgactactgcaagaatactaagaattggtactgctatgggaagacagtggcggaaaagacagcgcgggatgaggcaagggaaaagggagtggatttggttgtgattaatccagttttggtgcttggaccactgcttcaaccaacagtgaatgccagtgttcttcacatactaaagtacctaactggatctgctaaaacatatgccaattcaattcaggcgtatgttcatgttaaggatgtggcactagcccacatacttctctacgaagctccttctgcatctggccgctatatctgcgcggagcgcgtgcttcatcgcggtgatgtggttgaaattctcgccaaattcttcccggagtatccaatccccaccaagtgctcggatgaaacgaggccaagggcaaaaccgtacatattcacgaaccaaaagctaaaggacttgggtttggagtttacgccagtgaaacaatgcttatatgagacagtgaagagtctgcaggagaagggtcaccttccagttcctactcaaaatgatgaacctattaaaattcactcttaa

SEQ ID NO: 47 - protein, 332 aa, Lycopersicon esculentum
MPSESGKVVCVTGAGGFIASWLVKLLLEKGYTVRGTVRNPDDSKNGHLKELEGAKERLILLRADLLDYQSLREAIYGCDGVFHTASPVTDDPEQMVEPAVIGTKNVITAAAETKVRRVVFTSSIGTVYMDPNRAPDKVVDETCWSDLDYCKNTKNWYCYGKTVAEKTARDEAREKGVDLVVINPVLVLGPLLQPTVNASVLHILKYLTGSAKTYANSIQAYVHVKDVALAHILLYEAPSASGRYICAERVLHRGDVVEILAKFFPEYPIPTKCSDETRPRAKPYIFTNQKLKDLGLEFTPVKQCLYETVKSLQEKGHLPVPTQNDEPIKIHS

SEQ ID NO: 48 - DNA, 1032 bp, Panicum virgatum
atggccgtcaccgtgtgcgtcaccggcgccggcggcttcatcgggtcttggatcgtcaagctcctcctcgaccgcgggtacgccgtccggggcacctcccgccgcgcagatgaccccaagaacgcgcacctctgggcgctcgacggcgcggcggagcgcctcacgatgctgcaggtggacctgctcgaccgggccagcctccgcgccgccttcgacggctgcgacggcgtcatccacaccgcctcgccgatgcacgacaaccccgaggagatcatcgagccaattatcgccgggacgcggaacgtcgttgaggccgcggccgacgccggcgtgcggcgcctggtgatctcctccaccatcggcaccatgtacatgaacccgcaccgcgaccccgacgcgcccctggaggagtggagctggagcgacctcgagcactgcaagaagaccgcgaactggtactgctacgccaagacgatcgcggagcagagcgcgtggcaggcggcgcgggcgcggggcctggacctggcggtggtcatcccggtggtggtgctcggcgaactgatgcagccaagcatgaacaccagcaccctgcacatactcaagtacctcaccggccaggccaaggactatgtcaacgagtcgcacgcgtacgtgcacgtcaaggacgccgccgaggcgcacgtccaggtgctcgaggcacccggcgccggagggcatcgctacgtctgtgccgagcggactctgcaccgcggcgagctgtgccgcatgctcgctcaactcttcccggagtaccctattccgacgaggtgcaaggatgaggtgaatccaccaaagaagggctacaagtttacaaaccagcctctgaaggacctaggcatcaggttcacacctgcgcatgagtacctctatgaagcagtaaaatccctgcaggaaaaggattttctctccaaggcttctgtcacagaggtgactgaaagtagcagttccccgcctcaaaagttgcgactgacgacattgatttcaaacctttga

SEQ ID NO: 49 - protein, 343 aa, Panicum virgatum
MAVTVCVTGAGGFIGSWIVKLLLDRGYAVRGTSRRADDPKNAHLWALDGAAERLTMLQVDLLDRASLRAAFDGCDGVIHTASPMHDNPEEIIEPIIAGTRNVVEAAADAGVRRLVISSTIGTMYMNPHRDPDAPLEEWSWSDLEHCKKTANWYCYAKTIAEQSAWQAARARGLDLAVVIPVVVLGELMQPSMNTSTLHILKYLTGQAKDYVNESHAYVHVKDAAEAHVQVLEAPGAGGHRYVCAERTLHRGELCRMLAQLFPEYPIPTRCKDEVNPPKKGYKFTNQPLKDLGIRFTPAHEYLYEAVKSLQEKDFLSKASVTEVTESSSSPPQKLRLTTLISNL

SEQ ID NO: 50 - DNA, 1032 bp, Panicum virgatum
atggccgtcaccgtgtgcgtcaccggcgccggcggcttcatcgggtcttggatcgtcaagctcctcctcgaccgcgggtacgccgtccggggcacctcccgccgcgcagatgaccccaagaacgcgcacctctgggcgctcgacggcgcggcggagcgcctcacgatgctgcaggtggacctgctcgaccgggccagcctccgcgccgccttcgacggctgcgacggcgtcatccacaccgcctcgccgatgcacgacaaccccgaggagatcatcgagccaattatcgccgggacgcggaacgtcgttgaggccgcggccgacgccggcgtgcggcgcctggtgatctcctccaccatcggcaccatgtacatgaacccgcaccgcgaccccgacgcgcccctggaggagtggagctggagcgacctcgagcactgcaagaagaccgcgaactggtactgctacgccaagacgatcgcggagcagagcgcgtggcaggcggcgcgggcgcggggcctggacctggcggtggtcatcccggtggtggtgctcggcgaactgatgcagccaagcatgaacaccagcaccctgcacatactcaagtacctcaccggccaggccaaggactatgtcaacgagtcgcacgcgtacgtgcacgtcaaggacgccgccgaggcgcacgtccaggtgctcgaggcacccggcgccggagggcatcgctacgtctgtgccgagcggactctgcaccgcggcgagctgtgccgcatgctcgctcaactcttcccggagtaccctattccgacgaggtgcaaggatgaggtgaatccaccaaagaagggctacaagtttacaaaccagcctctgaaggacctaggcatcaggttcacacctgcgcatgagtacctctatgaagcagtaaaatccctgcaggaaaagggttttctctccaaggcttctgtcacagaggtgactgaaagtagcagttccccgcctcaaaagttgcgactgacgacattgatttcaaacctttga

SEQ ID NO: 51 - protein, 343 aa, Panicum virgatum
MAVTVCVTGAGGFIGSWIVKLLLDRGYAVRGTSRRADDPKNAHLWALDGAAERLTMLQVDLLDRASLRAAFDGCDGVIHTASPMHDNPEEIIEPIIAGTRNVVEAAADAGVRRLVISSTIGTMYMNPHRDPDAPLEEWSWSDLEHCKKTANWYCYAKTIAEQSAWQAARARGLDLAVVIPVVVLGELMQPSMNTSTLHILKYLTGQAKDYVNESHAYVHVKDAAEAHVQVLEAPGAGGHRYVCAERTLHRGELCRMLAQLFPEYPIPTRCKDEVNPPKKGYKFTNQPLKDLGIRFTPAHEYLYEAVKSLQEKGFLSKASVTEVTESSSSPPQKLRLTTLISNL

SEQ ID NO: 52 - DNA, 1032 bp, Panicum virgatum
atggccgtcaccgtgtgcgtcaccggcgccggcggcttcatcgggtcttggatcgtcaagctcctcctcgaccgcgggtacgccgtccggggcacctcccgccgcgcagatgaccccaagaacgcgcacctctgggcgctcgacagcgcggcggagcgcctcacgatgctgcaggtggacctgctcgaccgggccagcctccgcgccgccttcgacggctgcgacggcgtcatccacaccgcctcgccgatgcacgacaaccccgaggagatcatcgagccaattatcgccgggacacggaacgtcgtggaggccgcggccgacgccggcgtgcggcgcctggtgatctcttccaccatcggcaccatgtacatgaacccgcaccgcgaccccgacgcgcccctggacgagtggagctggagcgacctcgcgcactgcaagaagaccgcgaactggtactgctacgccaagacgatcgcggagcagagcgcgtggcaggcggcgcgggcgcggggcctggacctggcggtggtcatcccggtggtggtgctcggcgaactgatgcagccaagcatgaacaccagcaccctgcacatactcaagtacctcaccggccaggccaaggactatgtcaacgagtcgcacgcgtacgtgcacgtcaaggacgccgccgaggcgcacgtccgggtgctcgaggcacccggcaccggagggcatcgctacgtctgtgccgagcggactctgcaccgcggcgagctgtgccgcatgctcgctcaactcttcccggagtaccctattccgacgaggtgcaaggatggggtgaatccaccaaagaagggctacaagtttacaaaccagcctctgaaggacctaggcatcaggttcacacctacgcatgagtacctctatgaagcagtaaaatccctgcaggaaaagggttttctccccaaggcttctgtcacagaggtgactgaaagtagcagttccccgcctcaaaagttgcgactgacgacattgatttcaaacctttga

SEQ ID NO: 53 - protein, 343 aa, Panicum virgatum
MAVTVCVTGAGGFIGSWIVKLLLDRGYAVRGTSRRADDPKNAHLWALDSAAERLTMLQVDLLDRASLRAAFDGCDGVIHTASPMHDNPEEIIEPIIAGTRNVVEAAADAGVRRLVISSTIGTMYMNPHRDPDAPLDEWSWSDLAHCKKTANWYCYAKTIAEQSAWQAARARGLDLAVVIPVVVLGELMQPSMNTSTLHILKYLTGQAKDYVNESHAYVHVKDAAEAHVRVLEAPGTGGHRYVCAERTLHRGELCRMLAQLFPEYPIPTRCKDGVNPPKKGYKFTNQPLKDLGIRFTPTHEYLYEAVKSLQEKGFLPKASVTEVTESSSSPPQKLRLTTLISNL

SEQ ID NO: 54 - DNA, 840 bp, Panicum virgatum
atggccgtcaccgtgtgcgtcaccggcgccggcggcttcatcgggtcttggatcgtcaagctcctcctcgaccgcgggtacgccgtccggggcacctcccgccgcgcagatgaccccaagaacgcgcacctctgggcgctcgacggcgcggcggagcgcctcacgatgctgcaggtggacctgctcgaccgggcgcggggcctggacctggcggtggtcatcccggtggtggtgctcggcgaactgatgcagccaagcatgaacaccagcaccctgcacatactcaagtacctcaccggccaggccaaggactatgtcaacgagtcgcacgcgtacgtgcacgtcaaggacgccgccgaggcgcacgtccaggtgctcgaggcacccggcgccggagggcatcgctacgtctgtgccgagcggactctgcaccgcggcgagctgtgccgcatgctcgctcaactcttcccggagtaccctattccgacgaggtgcaaggatgaggtgaatccaccaaagaagggctacaagtttacaaaccagcctctgaaggacctaggcatcaggttcacacctgcgcatgagtacctctatgaagcagtaaaatccctgcaggaaaagggttttctctccaaggcttctgtcacagaggtaaaaaaaaaaaaggtgactgaaagtagcagttccccgcctcaaaagttgcgactgacgacattgatttcaacctttgaaagggggggcggccgcggagcctgcttttttgtacaaagttggcattataaaaaaagcattgctcatcaattttgttgcaacgaacaggtcactatcagtcaaaaataa

SEQ ID NO: 55 - protein, 279 aa, Panicum virgatum
MAVTVCVTGAGGFIGSWIVKLLLDRGYAVRGTSRRADDPKNAHLWALDGAAERLTMLQVDLLDRARGLDLAVVIPVVVLGELMQPSMNTSTLHILKYLTGQAKDYVNESHAYVHVKDAAEAHVQVLEAPGAGGHRYVCAERTLHRGELCRMLAQLFPEYPIPTRCKDEVNPPKKGYKFTNQPLKDLGIRFTPAHEYLYEAVKSLQEKGFLSKASVTEVKKKKVTESSSSPPQKLRLTTLISTFERGGGRGACFFVQSWHYKKSIAHQFCCNEQVTISQK

SEQ ID NO: 56 - DNA, 963 bp, Panicum virgatum
atggaggcggcggggaagagcgtgtgcgtgaccggcgcgggcggcttcatcgcgtcgtggctcgtgaaggtcctcctctcccggggctactactctgtgcgcggcaccgtgcgcgacccaggtgctagcaagaatgctcatctcaaggcgttggacggtgctggggaaaggttgcagcttctcaaagctgatttgttggactacaacagcgttgcatcagcggttgctggttgtgagggtgtcttccacgtagctagccctgttccctttggtcgatcctccaaccccgaggtggaagtcataggtcctgctgtcacaggtactgccaatgtgttaaaggcttcttatgaggcaaaagttggtagagttgtggtggtgtcttctattgcagctgtgtccaataatcctaactggcctaagggcaaggcttttgatgaagatagctggtcagatgaggagtactgcaggaagaatgaggattggtataacctctccaaaactctggcagaatgcgaggcctttgcttatgcagaaaaaacggggctagatgttgtaactatttgcccgtcattggtacttggacccttgatgcagtctacagtaaatgcaagcagtaaagtcctccttaattatttcaaaggggatcatgacaccgttgaaaatagactcagaaatattgtggatgttcgcgatgttactgatgctcttcttctggcatatgaaaaaccagaggcatctggacgatacatctgcagttcacatccaataaaagtttctgatatgatgaacatattgaagaacttatatccaacttacacttatcccaaaaactttgtggaagtggaagggaattttgtagacaattcggagaaacttcaaaagctaggatggaccttcaggccaatagaggaaacccttcgtgattgcgttgaatcctacaaaggatttggcttactaaattga

SEQ ID NO: 57 - protein, 320 aa, Panicum virgatum
MEAAGKSVCVTGAGGFIASWLVKVLLSRGYYSVRGTVRDPGASKNAHLKALDGAGERLQLLKADLLDYNSVASAVAGCEGVFHVASPVPFGRSSNPEVEVIGPAVTGTANVLKASYEAKVGRVVVVSSIAAVSNNPNWPKGKAFDEDSWSDEEYCRKNEDWYNLSKTLAECEAFAYAEKTGLDVVTICPSLVLGPLMQSTVNASSKVLLNYFKGDHDTVENRLRNIVDVRDVTDALLLAYEKPEASGRYICSSHPIKVSDMMNILKNLYPTYTYPKNFVEVEGNFVDNSEKLQKLGWTFRPIEETLRDCVESYKGFGLLN

SEQ ID NO: 58 - DNA, 1011 bp, Panicum virgatum
atgagctccgcggcggcggcgatgacgggggcggggaaggtggtgtgcgtgaccggcgcgtcggggtacatcgcgtcctggatcgtcaagctgcttctcgcccgcggatacaccgtccgtgccaccgtgcgggacaccgctgacccaaagaaaacattgcacctgagtgcgttggatggagctaaggataggctgcacttctttaaagcaagtctgctagaagagggatcttttgatgctgcagttgatggctgtgaaactgtgttccatactgcttctcccttttatcacaatgtcaaggatcctaaggctgagttacttgacccagcagtgaagggaacactcaatgttcttggttcatgcacgaaagcttctattaaaaaggtggttgtaacatcatccgttgctgctgttgcctacaatggaaagccaagaactcctgaagttatagttgatgagacatggttttctgatccacaaatttgtgaaaagaatcaacaatggtatgttctgtccaagacgcttgcagaggaggctgcttggaagttctctagggataacggattggaaattgttacgataaacccagcaatggttattggacccctgttgcagcctacactaaatactagtgctgaagcaatcctaaagctaatcaatgggtcatcgtctacatatcccaatttcagctttggatgggtgaatgttaaagatgttgcactggcacatatccttgcatatgaagttccctcagcacatggaagatattgcatggtggaaagagttgctcactactcagaagttgtcaacatcatacgcaagatgtatcctacaattcctcttgcagacaagtgtgcagatgacaaaccatttgtcccaacataccaggtatcaaaggagaaaataagaagcttaggcatcgagttgattccactggagatgtgcatcagggagaccatcgagagcttaaaagagaagggatttgttagttttgactatagcaatctgtga

SEQ ID NO: 59 - protein, 336 aa, Panicum virgatum
MSSAAAAMTGAGKVVCVTGASGYIASWIVKLLLARGYTVRATVRDTADPKKTLHLSALDGAKDRLHFFKASLLEEGSFDAAVDGCETVFHTASPFYHNVKDPKAELLDPAVKGTLNVLGSCTKASIKKVVVTSSVAAVAYNGKPRTPEVIVDETWFSDPQICEKNQQWYVLSKTLAEEAAWKFSRDNGLEIVTINPAMVIGPLLQPTLNTSAEAILKLINGSSSTYPNFSFGWVNVKDVALAHILAYEVPSAHGRYCMVERVAHYSEVVNIIRKMYPTIPLADKCADDKPFVPTYQVSKEKIRSLGIELIPLEMCIRETIESLKEKGFVSFDYSNL

SEQ ID NO: 60 - DNA, 963 bp, Panicum virgatum
atgagctccgcggcggcggcgatgacgggggcggggaaggtggtgtgcgtgaccggcgcgtcggggtacatcgcgtcctggatcgtcaagctgcttctcgcccgcggatacaccgtccgtgccaccgtgcgggacaccgctgacccaaagaaaacattgcacctgagtgcgttggatggagctaaggataggctgcacttctttaaagcaagtctgctagaagagggatcttttgatgctgcagttgatggctgtgaaactgtgttccatactgcttctcccttttatcacaatgtcaaggatcctaaggccgagttacttgacccagcagtgaagggaacactcaatgttcttggttcatgcacgaaagcttctattaaaaaggtggttgtaacatcatccgttgctgctgttgcctacaatggaaagccaagaactcctgaagttatagttgatgagacatggttttctgatccacaaatttgtgaaaagaatcaacaatggtatgttctgtccaagacgcttgcagaggaggctgcttggaagttctctagggataacggattggaaattgttacgataaacccagcaatggttattggacccctgttgcagcctacactaaatactagtgctgaagcaatcctaaagctaatcaatgggtcatcgtctacatatcccaatttcagctttggatgggtgaatgttaaagatgttgcactggcacatatccttgcatatgaagttccctcagcacatggaagatattgcatggtggaaagagttgctcactactcagaagttgtcaacatcatacgcaagatgtatcctacaattcctcttgcagacaagtgtgcagatgacaaaccatttgtcccaacataccaggtatcaaaggagaaaataagaagcttaggcatcaagttgattccactggagatgtgcatcagggagaccatctag

SEQ ID NO: 61 - protein, 320 aa, Panicum virgatum
MSSAAAAMTGAGKVVCVTGASGYIASWIVKLLLARGYTVRATVRDTADPKKTLHLSALDGAKDRLHFFKASLLEEGSFDAAVDGCETVFHTASPFYHNVKDPKAELLDPAVKGTLNVLGSCTKASIKKVVVTSSVAAVAYNGKPRTPEVIVDETWFSDPQICEKNQQWYVLSKTLAEEAAWKFSRDNGLEIVTINPAMVIGPLLQPTLNTSAEAILKLINGSSSTYPNFSFGWVNVKDVALAHILAYEVPSAHGRYCMVERVAHYSEVVNIIRKMYPTIPLADKCADDKPFVPTYQVSKEKIRSLGIKLIPLEMCIRETI

SEQ ID NO: 62 - DNA, 984 bp, Panicum virgatum
atgagctccgcggcggcggcgatgacgggggcggggaaggtggtgtgcgtgaccggcgcgtcggggtacatcgcgtcctggatcgtcaagctgcttctcgcccgcggatacaccgtccgtgccaccgtgcgggacaccgctgacccaaagaaaacattgcacctgagtgcgttggatggagctaaggataggctgcacttctttaaagcaagtctgctagaagagggatcttttgatgctgcagttgatggctgtgaaactgtgttccatactgcttctcccttttatcacaatgtcaaggatcctaaggctgagttacttgacccagcagtgaagggaacactcaatgttcttggttcatgcacgaaagcttctattaaaaaggtggttgtaacattatccgttgctgctgttgcctacaatggaaagccaagaactcctgaagttatagttgatgagacatggttttctgatccacaaatttgtgaaaagaatcaacaatggtatgttctgtccaagacgcttgcagaggaggctgcttggaagttctctagggataacggattggaaattgttacgataaacccagcaatggttattggacccctgttgcagcctacactaaatactagtgctgaagcaatcctaaagctaatcaatgggtcatcgtctacatatcccaatttcagctttggatgggtgaatgttaaagatgttgcactggcacatatccttgcatatgaagttccctcagcacatggaagatattgcatggtggaaagagttgctcactactcagaagttgtcaacatcatacgcaagatgtgtgcagatgacaaaccatttgtcccaacataccaggtatcaaaggagaaaataagaagcttaggcatcgagttgattccactggagatgtgcatcagggagaccatcgagagcttaaaagagaagggatttgttagttttgactatagcaatctgtga

SEQ ID NO: 63 - protein, 327 aa, Panicum virgatum
MSSAAAAMTGAGKVVCVTGASGYIASWIVKLLLARGYTVRATVRDTADPKKTLHLSALDGAKDRLHFFKASLLEEGSFDAAVDGCETVFHTASPFYHNVKDPKAELLDPAVKGTLNVLGSCTKASIKKVVVTLSVAAVAYNGKPRTPEVIVDETWFSDPQICEKNQQWYVLSKTLAEEAAWKFSRDNGLEIVTINPAMVIGPLLQPTLNTSAEAILKLINGSSSTYPNFSFGWVNVKDVALAHILAYEVPSAHGRYCMVERVAHYSEVVNIIRKMCADDKPFVPTYQVSKEKIRSLGIELIPLEMCIRETIESLKEKGFVSFDYSNL

SEQ ID NO: 64 - DNA, 963 bp, Panicum virgatum; identical to SEQ ID NO: 60

SEQ ID NO: 65 - protein, 320 aa, Panicum virgatum; identical to SEQ ID NO: 61

SEQ ID NO: 66 - DNA, 1095 bp, Panicum virgatum
atgaccgtggttgacgccgtgtccgcgggcgccggcgacgccgccgccgcggtgccgcagccggcggggaacgggcagaccgtgtgcgtcaccggcgcggccgggtacatcgcgtcgtggctcgtcaagctgctgctcgagaaggggtacactgtcaagggcaccgtcaggaacccagatgacccgaagaacgcgcacctcaaggcgctggacggcgccgccgagcggctcatcctctgcaaggccgacctcctggactacgacgccatctgccgcgccgtggagggctgccacggcgtcttccacaccgcctcccccgtcaccgacgaccccgagcaaatggtggagccggcggtgcgcggcacggagtacgtgatcagcgcggcggcggaggccggcacggtacggcgggtggtgttcacgtcctccatcggcgccgtgaccatggaccccaaccgcgggcccgacgtggtggtcgacgagtcctgctggagcgacctcgacttctgcaagaaaaccaggaactggtactgctacggcaaggcggtggcggagcaggcggcgtgggaggcggcgcggcagcgcggcgtggacctggtggtggtgaacccggtgctggtggtgggcccgctgcagcagccgacggtgaacgccagcatcgcgcacatcctcaagtacctggacggctccgcccgcaccttcgccaacgccgtgcaggcctacgtcgacgtccgcgacgtcgccgccgcgcacctccgcgtcttccagagccccgccgcctccggccgccacctctgcgccgagcgcgtcctccaccgcgaggacgtcgtccgcatcctcgccaagctcttccccgagtaccccgtccccaccaggtgctccgacgaggtgaacccgcggaagcaggcgtacaagtttacgaaccagaagctccgggacctggggctggagttccggccggtgagccagtcgctgtacgacacggtgaagagcctccaggagaaggaccacctgccggtgctcgccgacgagcaggcgccggaggccgcccccggcgccgaggcgcaacagggcgggatcgccatccgtgcgtga

SEQ ID NO: 67 - protein, 364 aa, Panicum virgatum
MTVVDAVSAGAGDAAAAVPQPAGNGQTVCVTGAAGYIASWLVKLLLEKGYTVKGTVRNPDDPKNAHLKALDGAAERLILCKADLLDYDAICRAVEGCHGVFHTASPVTDDPEQMVEPAVRGTEYVISAAAEAGTVRRVVFTSSIGAVTMDPNRGPDVVVDESCWSDLDFCKKTRNWYCYGKAVAEQAAWEAARQRGVDLVVVNPVLVVGPLQQPTVNASIAHILKYLDGSARTFANAVQAYVDVRDVAAAHLRVFQSPAASGRHLCAERVLHREDVVRILAKLFPEYPVPTRCSDEVNPRKQAYKFTNQKLRDLGLEFRPVSQSLYDTVKSLQEKDHLPVLADEQAPEAAPGAEAQQGGIAIRA

SEQ ID NO: 68 - DNA, 1095 bp, Panicum virgatum
atgaccgtggttgacgccgtgtccgcgggcgccggcgacgccgccgccgcggtgccgcagccggcggggaacgggcagaccgtgtgcgtcaccggcgcggccgggtacatcgcgtcgtggctcgtcaagctgctgctcgagaaggggtacactgtcaagggcaccgtcaggaacccagatgacccgaagaacgcgcacctcaaggcgctggacggcgccgccgagcggctcatcctctgcaaggccgacctcctggactacgacgccatctgccgcgccgtggagggctgccacggcgtcttccacaccgcctcccccgtcaccgacgaccccgagcaaatggtggagccggcggtgcgcggcacggagtacgtgatcagcgcggcggcggaggccagcacggtacggcgggtggtgttcacgtcctccatcggcgccgtgaccatggaccccaaccgcgggcccgacgtggtggtcgacgagtcctgctggagcgacctcgacttctgcaagaaaaccaggaactggtactgctacggcaaggcggtggcggagcaggcggcgtgggaggcggcgcggcagcgcggcgtggacctggtggtggtgaacccggtgctggtggtgggcccgctgcagcagccgacggtgaacgccagcatcgcgcacatcctcaagtacctggacggctccgcccgcaccttcgccaacgccgtgcaggcctacgtcgacgtccgcgacgtcgccgccgcgcacctccgcgtcttccagagccccgccgcctccggccgccacctctgcgccgagcgcgtcctccaccgcgaggacgtcgtccgcatcctcgccaagctcttccccgagtaccccgtccccaccaggtgctccgacgaggtgaacccgcggaagcaggcgtacaagtttacgaaccagaagctccgggacctggggctggagttccggccggtgagccagtcgctgtacgacacggtgaagagcctccaggagaaggaccacctgccggtgctcgccgacgagcaggcgccggaggccgcccccggcgccgaggcgcaacagggcgggatcgccatccgtgcgtga

SEQ ID NO: 69 - protein, 364 aa, Panicum virgatum
MTVVDAVSAGAGDAAAAVPQPAGNGQTVCVTGAAGYIASWLVKLLLEKGYTVKGTVRNPDDPKNAHLKALDGAAERLILCKADLLDYDAICRAVEGCHGVFHTASPVTDDPEQMVEPAVRGTEYVISAAAEASTVRRVVFTSSIGAVTMDPNRGPDVVVDESCWSDLDFCKKTRNWYCYGKAVAEQAAWEAARQRGVDLVVVNPVLVVGPLQQPTVNASIAHILKYLDGSARTFANAVQAYVDVRDVAAAHLRVFQSPAASGRHLCAERVLHREDVVRILAKLFPEYPVPTRCSDEVNPRKQAYKFTNQKLRDLGLEFRPVSQSLYDTVKSLQEKDHLPVLADEQAPEAAPGAEAQQGGIAIRA

SEQ ID NO: 70 - DNA, 1092 bp, Panicum virgatum
atgaccgtggttgacgccgtgtccgcgggcgccggcgacgccgccgccgcggtgccgcagccggcggggaacgggcagaccgtgtgcgtcaccggcgcggccgggtacatcgcgtcgtggctcgtcaagctgctgctcgagaaggggtacactgtcaagggcaccgtcaggaacccagatgacccgaagaacgcgcacctcaaggcgctggacggcgccgccgagcggctcatcctctgcaaggccgacctcctggactacgacgccatctgccgcgccgtggagggctgccacggcgtcttccacaccgcctcccccgtcaccgacgaccccgagcaaatggtggagccggcggtgcgcggcacggagtacgtgatcagggcggcggcggaggccggcacggtgcggcgggtggtgttcacgtcctccatcggcgccgtcaccatggaccccaaccgcgggcccgacgtggtggtcgacgaatcctgctggagcgacctcgacttctgcaagaaaaccaggaactggtactgctacggcaaggcggtggcggagcaggcggcgtgggatgcggcgcggcagcgcggcgtggacctggtggtggtgaacccggtgctggtggtgggcccgctgctgcagccgacggtgaacgccagcatcgcgcacatcctcaagtacctggacggctccgcccgcaccttcgccaacgccgtgcaggcctacgtcgacgtccgcgacgtcgccgccgcgcacctcctcgtcttcgaggcccccgccgcctccggccgccacctctgcgccgaccgcgtcctccaccgcgaggacgtcgtccgcatcctcgccaagctcttccccgagtaccccgtccccaccaggtgctccgacgaggtgaacccgcggaagcaggcgtacaagttctcgaaccagaagctccgggacctggggctggagttccggccggtgagccagtcgctgtacgacacggtgaagagcctccaggagaagggccacctgccggtgctcgccgagcaggcgccggaggccgcccccggcgccgaggcgcagcagggcgggatcgccatccgtgcgtga

SEQ ID NO: 71 - protein, 363 aa, Panicum virgatum
MTVVDAVSAGAGDAAAAVPQPAGNGQTVCVTGAAGYIASWLVKLLLEKGYTVKGTVRNPDDPKNAHLKALDGAAERLILCKADLLDYDAICRAVEGCHGVFHTASPVTDDPEQMVEPAVRGTEYVIRAAAEAGTVRRVVFTSSIGAVTMDPNRGPDVVVDESCWSDLDFCKKTRNWYCYGKAVAEQAAWDAARQRGVDLVVVNPVLVVGPLLQPTVNASIAHILKYLDGSARTFANAVQAYVDVRDVAAAHLLVFEAPAASGRHLCADRVLHREDVVRILAKLFPEYPVPTRCSDEVNPRKQAYKFSNQKLRDLGLEFRPVSQSLYDTVKSLQEKGHLPVLAEQAPEAAPGAEAQQGGIAIRA

SEQ ID NO: 72 - protein, 8 aa, Panicum virgatum
KNWYCYGK

SEQ ID NO: 73 - protein, 342 aa, Panicum virgatum
MPAATAAAAAESSSVSGETICVTGAGGFIASWMVKLLLEKGYTVRGTLRNPDDPKNGHLKKLEGAKERLTLVKVDLLDLNSVKEAVNGCHGVFHTASPVTDNPEEMVEPAVNGAKNVIIAGAEAKVRRVVFTSSIGAVYMDPNRSVDVEVDESCWSDLEFCKKTKNWYCYGKAVAEAAAWDVAKEKGVDLVVVNPVLVLGPLLQPTINASTIHILKYLTGSAKTYANATQAYVHVRDVALAHILVYEKPSASGRYLCAETSLHRGELVEILAKYFPEYPIPTKCSDEKNPRVKPHIFSNKKLKDLGLEFTPVSECLYETVKSLQDQGHLSIPNKEDSLAVKS

(19) United States
(12) Patent Application Publication
(10) Pub. No.: US 2012/0047600 A1
(43) Pub. Date: Feb. 23, 2012

(54) CAFFEOYL COA REDUCTASE

(76) Inventors: Rui ZHOU, Ardmore, OK (US); Richard A. DIXON, Sulphur, OK (US); Fang CHEN, Ardmore, OK (US)

(21) Appl. No.: 13/181,284

(22) Filed: Jul. 12, 2011

Related U.S. Application Data
(60) Provisional application No. 61/363,556, filed on Jul. 12, 2010.

(51) Int. Cl. A01H 1/06; C12N 15/63; C12N 15/82; C12N 9/10; A01H 5/00; A01H 5/10; C12N 5/10; C08H 7/00; C07C 29/74; C07G 99/00; A01H 5/06; A01H 5/02; A23K 1/00; C01B 3/32; D21C 5/00; D01F 9/12; C12N 15/54

(52) U.S. Cl. 800/278; 536/23.2; 435/320.1; 435/193; 800/298; 800/320.1; 800/317.4; 800/312; 800/320; 435/419; 530/500; 568/913; 44/307; 426/635; 48/197 R; 162/96; 162/97; 162/99; 423/447.1

(57) ABSTRACT

The invention provides methods for increasing lignin content in plants by expression of a cinnamoyl CoA reductase 2 (CCR2) coding sequence in the plant. Also provided are methods for reducing lignin content in a plant by down-regulation of CCR2 expression in the plant. Nucleic acid molecules for modulation of CCR2 expression and transgenic plants the same are also provided. Plants described herein may be used, for example, as improved biofuel feedstock and as highly digestible forage crops. Methods for processing plant tissue and for producing biofuels by utilizing such plants are also provided.

### CROSS-REFERENCE TO RELATED APPLICATIONS

This application claims the benefit of priority of U.S. Provisional Application Ser. No. 61/363,556, filed on Jul. 12, 2010, the disclosure of which is incorporated herein by reference in its entirety.

### STATEMENT OF GOVERNMENTAL INTEREST

This invention was made with Government support under DE-FG02-06ER64303 awarded by the U.S. Department of Energy. The Government has certain rights in the invention.

### BACKGROUND OF THE INVENTION

### Incorporation by Reference of Sequence Listing in Computer Readable Form

The Sequence Listing, which is a part of the present disclosure, includes a computer readable form 88 kb file entitled “NBLE074US_ST25.TXT” comprising nucleotide and/or amino acid sequences of the present invention submitted via EFS-Web. The subject matter of the Sequence Listing is incorporated herein by reference in its entirety.

1. Field of the Invention

The present invention relates generally to the field of agriculture and plant genetics. More particularly, it concerns genetically modified plants comprising altered lignin content.

2. Description of Related Art

Modification of plant biomass content has recently become an intense area of research due to the broad ranging commercial applications for such technology. For example, biofuel is increasingly being considered as a renewable, cleaner alternative to petroleum-based fuels. A variety of fuels may also be produced from sugars and starches as well as from lignocellulosic based biomass, which constitute the most abundant biomass on earth. However, the types of biofuels that can be efficiently produced from plant mass depend upon the content of component material such as lignin.

Likewise, biomass content dictates the nutritional value of plant mass as animal feed. In particular, high lignin content in plant matter can result in animal feed that is difficult for livestock to digest.

Development of plants with modified cell wall composition would have a significant benefit for the production of biofuels and animal feeds and could potentially have a broad range of other beneficial applications. In some instances, increasing lignin would increase the energy content of biomass for gasification, and would also lead to increased carbon sequestration. However genetic modification of plants to achieve these goals has not been realized.

### SUMMARY OF THE INVENTION

In a first embodiment, there is provided a plant or plant cell comprising a CCR2 coding sequence operably linked to a heterologous promoter wherein the plant exhibits increased lignin content. For example, a CCR2 coding sequence may encode a polypeptide having at least 60%, 70%, 80%, 85%, 90%, 95%, 98%, 99% or greater amino acid identity to SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71 having caffeoyl CoA reductase activity.

In a further embodiment, there is provided a nucleic acid molecule for expression of CCR2 comprising a nucleic acid sequence encoding a CCR2 coding sequence operably linked to a heterologous promoter. For example, a nucleic acid may be selected from the group consisting of: (a) a nucleic acid sequence encoding the polypeptide sequence of SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71; (b) a nucleic acid sequence comprising a sequence selected from the group consisting of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70; (c) a nucleic acid sequence that hybridizes to SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70, under conditions of 1×SSC, and 65° C. and encodes a polypeptide with caffeoyl CoA reductase activity; (d) a nucleic acid sequence encoding a polypeptide with at least 85%, 90%, 95%, 96%, 97%, 98% or 99% amino acid identity to SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71, having caffeoyl CoA reductase activity; (e) a nucleic acid sequence with at least 85%, 90%, 95%, 96%, 97%, 98% or 99% identity to SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70 and encodes a polypeptide with caffeoyl CoA reductase activity; and (f) a complement of a sequence of (a)-(e) wherein the nucleic acid sequence is operably linked to a heterologous promoter. In some aspects, a nucleic acid molecule provided comprises a nucleic acid sequence encoding a polypeptide having at least 85%, 90%, 95%, 96%, 97%, 98% or 99% amino acid sequence identity to the sequence of SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71, having caffeoyl CoA reductase activity, wherein the encoded polypeptide comprises the highly conserved CCR2 amino acid motif of SEQ ID NO: 72. Thus, in certain aspects, a nucleic acid molecule provided herein may be defined as nucleic acid molecule capable of expressing a functional CCR2 enzyme and thereby increasing lignin content in a plant expressing the sequence.

In still a further embodiment, there is provided a plant comprising down-regulated CCR2 gene expression wherein the plant exhibits reduced lignin content. As used herein, the term CCR2 gene refers to the CCR2 gene from M. truncatula and homologs thereof. For example, a homolog may be defined as a gene encoding a polypeptide having at least 60%, 70%, 80%, 85%, 90%, 95%, 98%, 99% or greater amino acid identity to SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71.

In one embodiment, a plant according to the invention comprises a selected DNA that down-regulates CCR2 gene expression. For example, a selected DNA may be defined as a genomic CCR2 sequence comprising a mutation that disrupts the gene by down-regulating CCR2 expression, by abrogating expression entirely or by rendering the gene product non-functional. For example, the mutation may be a point mutation, an insertion or a deletion and the mutation may be located in a coding (e.g., in a CCR2 exon) or non-coding potion to the CCR2 gene (e.g., in the CCR2 promoter region). Mutations in an CCR2 gene can be accomplished by any of the methods well known to those in the art including random mutagenesis methods such as irradiation, random DNA integration (e.g., via a transposon) or by using a chemical mutagen. Moreover, in certain aspects, a CCR2 gene may be mutated using a site-directed mutagenesis approach such as by using homologous recombination vector. Further detailed methods for inducing a mutations in plant genes are provided below.

In a further embodiment, a selected DNA that down-regulates CCR2 expression comprises a DNA molecule capable of expressing a nucleic acid sequence complementary to all or a portion of a CCR2 gene sequence or a CCR2 messenger RNA (mRNA). Thus, in some aspects, a transgenic plant may comprise an antisense, RNAi or miRNA construct for down-regulation of CCR2. For example, a transgenic plant can comprise a promoter which expresses a sequence complimentary to all or a portion of a CCR2 sequence from the plant. In certain specific embodiments, a transgenic plant comprises a nucleic acid molecule capable of expressing an nucleic acid sequence complementary to all or a portion of a Medicago CCR2 (SEQ ID NO:36), a Poplar CCR2 (SEQ ID NO:38; SEQ ID NO:40; SEQ ID NO:42; or SEQ ID NO:44), a tomato CCR2 (SEQ ID NO:46), or a switchgrass CCR2 (SEQ ID NO:48; SEQ ID NO:50; SEQ ID NO:52; SEQ ID NO:54; SEQ ID NO:56; SEQ ID NO:58; SEQ ID NO:60; SEQ ID NO:62; SEQ ID NO:64; SEQ ID NO:66; SEQ ID NO:68; or SEQ ID NO:70) nucleic acid sequence. Moreover, in certain aspects, the selected DNA that down-regulates CCR2 expression may comprise a tissue specific or inducible promoter operably linked to the nucleic acid sequence complimentary to all or part of a plant CCR2 gene or mRNA. In some cases, the promoter sequence is selected from the group consisting of a developmentally-regulated, organelle-specific, inducible, tissue-specific, constitutive, cell-specific, seed-specific, or germination-specific promoter.

In still further aspects there is provided a nucleic acid molecule comprising a nucleic acid sequence that when expressed in a cell down-regulates expression of CCR2 gene. For example, in certain aspects a nucleic acid is selected from the group consisting of: (a) a nucleic acid sequence that hybridizes to the nucleic acid sequence complementary to the sequence of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70, under conditions of 1×SSC and 65° C.; (b) a nucleic acid comprising the sequence of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70 or a fragment thereof; and (c) a nucleic acid sequence having at least 80% sequence identity to the nucleic acid sequence of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70; wherein the nucleic acid sequence is operable linked to a heterologous promoter sequence and wherein expression of the nucleic acid molecule in a plant cell reduces a CCR2 gene expression relative to an identical cell lacking the nucleic acid sequence. In some aspects, a DNA molecule provided comprises a complement of a fragment of a nucleic acid sequence encoding a CCR2 gene. For example, the nucleic acid fragment may be complementary to at least 17, 18, 19, 20, 21, 22, 23, 24, 25, 50, 100, 150, 200 or more nucleotides of a CCR2 gene sequence. Thus, in certain aspects, a nucleic acid molecule can be used to down-regulate a CCR2 gene and thereby reduce lignin content in a plant expressing the nucleic acid sequence.

In still further aspects, a transgenic plant further comprises a second DNA sequence that down-regulates lignin biosynthesis. For example, in certain embodiments, the second DNA sequence down-regulates a lignin biosynthesis gene selected from the group consisting of 4-coumarate 3-hydroxylase (C3H), phenylalanine ammonia-lyase (PAL), cinnamate 4-hydroxylase (C4H), hydroxycinnamoyl transferase (HCT), caffeic acid O-methyltransferase (COMT), caffeoyl CoA 3-O-methyltransferase (CCoAOMT), ferulate 5-hydroxylase (F5H), cinnamyl alcohol dehydrogenase (CAD), cinnamoyl CoA-reductase 1 (CCR1), 4-coumarate-CoA ligase (4CL), monolignol-lignin-specific glycosyltransferase, and aldehyde dehydrogenase (ALDH). In certain aspects, the second DNA comprises a mutated genomic copy of one or more lignin biosynthesis gene that disrupts expression of the gene or the function of the gene product. In still further aspects, a transgenic plant may further comprise a selected DNA that is an antisense or RNAi construct comprising an expressible nucleic acid sequence complimentary to all or part of a lignin biosynthesis gene. In certain embodiments, at least two, at least three or at least four additional lignin biosynthesis genes are down-regulated.

A variety of plants can be modified in accordance with the instant disclosure. For example, in some aspects, a plant may be a forage plant, a biofuel crop, a cereal crop or an industrial plant. For example, a forage plant may be a forage soybean, alfalfa, clover, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or reed canarygrass plant. In certain aspects, a plant is a biofuel crop including, but not limited to, switchgrass (Panicum virgatum), giant reed (Arundo donax), reed canarygrass (Phalaris arundinacea), Miscanthus x giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), soybeans, alfalfa, tomato, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or poplar. Cereal crops for use according to the instant disclose include, but are not limited to, maize, rice, wheat, barley, sorghum, millet, oat, rye, triticle, buckwheat, fonio or quinoa. In certain specific aspects, the plant may be defined as a Medicago, poplar, tomato or switchgrass plant having a CCR2 coding region of SEQ ID NO:36 (Medicago), SEQ ID NO:38; SEQ ID NO:40; SEQ ID NO:42; or SEQ ID NO:44 (Poplar), SEQ ID NO:46 (tomato), or SEQ ID NO:48; SEQ ID NO:50; SEQ ID NO52; SEQ ID NO:54; SEQ ID NO:56; SEQ ID NO:58; SEQ ID NO:60; SEQ ID NO:62; SEQ ID NO:64; SEQ ID NO:66; SEQ ID NO:68; or SEQ ID NO:70 (switchgrass).

In still further aspects there is provided a part of a plant described herein such as a protoplast, cell, meristem, root, pistil, anther, flower, seed, embryo, stalk or petiole.

In another embodiment, a transgenic or mutated plant produced herein may be further defined as an R0 plant, or as a progeny plant of any generation of an R0 plant, wherein the plant has inherited the selected DNA or mutation from the R0 plant. Moreover, in certain aspects, a progeny plant as described herein may be defined as a progeny plant that has been crossed with a second plant, such as a variety with reduced lodging. In other embodiments, the invention comprises a seed of a plant wherein the seed comprises a mutation or selected DNA described herein. A transgenic cell of such a plant also comprises an embodiment of the invention.

In further embodiments a transgenic plant, plant part or plant cell comprising a nucleic acid molecule as described herein is provided. For example, in certain aspects, nucleic acid molecules are provided that down-regulate CCR2 expression. Plants and plant parts comprising a down-regulated CCR2 may, in certain aspects, be defined as comprising decreased lignin content and increased fermentable carbohydrate content. In certain aspects, such plants may be used as feedstock for biofuel production. In another example, nucleic acid molecules are provided for expression or overexpression of CCR2. Plants and plant parts comprising CCR2 expression may, in certain aspects, be defined as comprising increased lignin content and may be used as biofuel feedstock for processes such as gasification.

In yet a further embodiment, there is provided a method of increasing the lignin content in a plant comprising expressing a CCR2 gene expression in the plant. Thus, in certain aspects a plant provided here may be defined as having increased lignin content relative to a wild-type counterpart. Moreover, in certain aspects a plant may be defined as having increased G lignin content or increased S lignin content. Plants provided herein comprising increased lignin content may, in certain aspects, be used in the manufacture of biofuel feedstock, carbon fibers derived from lignin or paper pulp materials.

Moreover, there is provided herein a method of decreasing the lignin content in a plant comprising down-regulating CCR2 gene expression in the plant. Thus, in certain aspects a plant provided here may be defined as having reduced lignin content relative to a wild-type counterpart. Moreover, in certain aspects a plant may be defined as having reduced G lignin content or reduced S lignin content. Plants provided herein comprising reduced lignin content may, in certain aspects, be used in the manufacture of biofuel feedstock (e.g., ethanol, butanol and biodiesel) or paper pulp materials.

In still a further embodiment, there is provided a method for increasing the digestibility of a forage crop comprising down-regulating CCR2 gene expression in the plant. For example, in certain aspects, plants described herein comprise reduced lignin content and have enhanced digestibility. In some cases such plants or parts thereof may be used for livestock forage or in the manufacture of a livestock feed.

In still a further embodiment, there is provided a method for the manufacture of a commodity product comprising obtaining a plant or plant part comprising a mutation or a selected DNA that down-regulates a CCR2 gene and producing a commercial product therefrom. For example, a plant or plant part described herein can be manufactured into a product such as, paper, paper pulp, ethanol, biodiesel, silage, animal feed or fermentable biofuel feedstock.

In yet another aspect, the invention provides a method of producing ethanol comprising: (a) obtaining a plant of a biofuel crop species comprising a selected DNA that down-regulates CCR2 gene expression in the plant wherein the plant exhibits an increase in fermentable carbohydrates relative to a plant of the same genotype lacking the selected DNA; (b) treating tissue from the plant to render carbohydrates in the tissue fermentable; and (c) fermenting the carbohydrates to produce ethanol.

In yet another aspect, the invention provides a method for processing lignocellulosic biomass from a plant or plant part described herein. In one embodiment the method for processing lignocellulosic biomass from a plant or plant part, may comprise acid and/or enzymatic treatment(s). The enzymatic treatment may comprise treatment with one or more cellulolytic enzymes, such as a cellulase. In another embodiment, the method comprises an acid treatment prior to or during a treatment to render carbohydrates in the plant fermentable. In yet another embodiment, no acid treatment is performed.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan however these terms may be used interchangeably with “comprise” or “comprising” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Depicts the currently accepted pathway for monolignol biosynthesis. The pathway as far as it pertains to coniferaldehyde is essentially linear. The enzymes are: PAL, L-phenylalanine ammonia-lyase; C4H, cinnamate 4-hydroxylase; 4CL, 4-coumarate: CoA ligase; CCR, cinnamoyl CoA reductase; CAD, cinnamyl alcohol dehydrogenase; HCT, hydroxycinnamoyl CoA: shikimate hydroxycinnamoyl transferase; C3H, “coumarate 3-hydroxylase” (more correctly termed coumaroyl shikimate 3-hydroxylase); CCoAOMT, caffeoyl CoA 3-O-methyltransferase; FSH, “ferulate 5-hydroxylase” (more correctly termed coniferaldehde 5-hydroxyalse); COMT, “caffeic acid 3-O-methyltransferase” (now designated 5-hydroxyconiferaldehye 5-O-methyltransferase).

FIG. 2A-D: Maule staining of syringyl lignin in stem cross sections from control (null segregant) alfalfa and plants down-regulated in expression of COMT, CCoAOMT or both. FIG. 2A, corresponds to null line #16; FIG. 2B, corresponds to COMT RNAi, line #10; FIG. 2C, corresponds to CCoAOMT RNAi line #37; and FIG. 2D, corresponds to double COMT/CCoAOMT RNAi line #25.

FIG. 3A-B: Graphs show lignin content and composition (internodes 1-7) of control and single/double O-methyltransferase down-regulated alfalfa lines. FIG. 3A, Acetyl bromide lignin content. FIG. 3B, Thioacidolysis monomer yields and lignin composition. S=syringyl monomers; G=guaiacyl monomers; H=hydroxyphenyl monomers. Results are average values of two analytical replicates. Maximum variance for acetyl bromide measurements is less than 5.6%, less than 6.2% for thioacidolysis.

FIG. 4: Amino acid sequence alignments of Medicago CCR1 (SEQ ID NO: 73) and CCR2 (SEQ ID NO: 32). The asterisks under the sequences indicate the conserved KNWYCYGK (SEQ ID NO: 72) motif.

FIG. 5A-F: Graphs show results of a functional analysis of recombinant Medicago CCRs expressed in E. coli. FIG. 5A-D, Initial velocity versus substrate concentration curves with caffeoyl CoA (FIG. 5A), coumaroyl CoA (FIG. 5B), feruloyl CoA (FIG. 5C) or sinapoyl CoA (FIG. 5D) as substrates. FIG. 5E-F, Hill plots for CCR2 with caffeoyl CoA and coumaroyl CoA as substrates, respectively.

FIG. 6A-B: Graphs show tissue-specific expression of CCR transcripts in M. truncatula. Transcript levels in flower, leaf and stem internodes 2, 4, 6 and 8 as determined by qRTPCR. Levels are expressed relative to ubiquitin. Error bars are the SD (standard deviation) of three replicates.

FIG. 7A-G: Characterization of retrotransposon insertion lines in Medicago CCR1. FIG. 7A, Positions of independent Tnt1 insertions in CCR1. FIG. 7B, RT-PCR analysis of CCR1 and Actin transcript levels in wild-type and CCR1 insertion lines. FIG. 7C, Growth of wild type (WT) Medicago R108 and ccr1-1 (left panels) and ccr1-2 (right panel). Plants in the upper left panel were 4 weeks post-germination, 10 weeks post-germination in the other panels. FIG. 7D, Extractable activities of CCR1 (with feruloyl CoA) in stem extracts from wild type and ccr1 mutant lines. The results are expressed as a percentage of the average of the wild type value. FIG. 7E, UV autofluorescence (panels a-c), phloroglucoinol staining (panels d-f) and Maule staining (panels g-i) of stem cross sections of wild type (panels a,d,g), ccr1-1 (panels b,e,h) and ccr1-2 (panels c,f,i). FIG. 7F, Acetyl bromide lignin levels in internodes 6 and 7 (counting from the top) of stems of wild type and ccr1-1 and ccr1-2 lines, harvested at the early flowering stage. FIG. 7G, Lignin thioacidolysis yields and monomer compositions of internodes 6 and 7 (counting from the top) of stems of wild type and ccr1-1 and ccr1-2 lines harvested at the early flowering stage. In all cases error bars represent SD of three replicates.

FIG. 8A-E: Characterization of retrotransposon insertion lines in Medicago CCR2. FIG. 8A, Positions of independent Tnt1 insertions in CCR2. FIG. 8B, RT-PCR analysis of CCR2 and Actin transcript levels in wild-type and CCR2 insertion lines. FIG. 8C, Extractable activities of CCR1 (with feruloyl CoA) and CCR2 (with caffeoyl CoA) in stem extracts from wild type and CCR2 mutant lines. Results are expressed as a percentage of the average value of wild type. FIG. 8D, Acetyl bromide lignin levels in internodes 6 and 7 of stems of wild type and ccr2-1, ccr2-2 and ccr2-3 lines harvested at early flowering. FIG. 8E, Lignin thioacidolysis yields and monomer compositions of internodes 6 and 7 of stems of wild type and ccr2-1, ccr2-2 and ccr2-3 lines harvested at early flowering. In all case error bars represent SD of five replicates.

FIG. 9A-F: Complementation of the Arabidopsis irx4 mutant with Medicago CCR1 and CCR2. FIG. 9A, RT-PCR screening to show expression of the Medicago transgenes in the irx4 background. FIG. 9B, Visible appearance of plants at 20 days post-germination. FIG. 9C, Extractable activities of CCR measured with feruloyl CoA and caffeoyl CoA in stem extracts from Arabidopsis ecotype Landsberg erecta (Ler), the irx4 mutant, and in irx4 complemented with Medicago CCR1 or CCR2. Results are expressed as a percentage of the average of that of Ler. FIG. 9D, UV autofluorescence (panels a-d), phloroglucoinol staining (panels e-h) and Maule staining (panels i-1) of stem cross sections of Arabidopsis Col-0 (panels a,e,i), irx4 (panels b,fj) and irx4 expressing CCR1 (panels c,g,k) or CCR2 (panels d,h,l). FIG. 9E, Acetyl bromide lignin levels in the inflorescence stems of Ler, the irx4 mutation in the Ler background, and in irx4 mutants complemented with Medicago CCR1 or CCR2, harvested at 25 day post germination. FIG. 9F, Lignin thioacidolysis yields and monomer compositions of the inflorescence stems of Ler, the irx4 mutation in the Ler background, and in irx4 mutants complemented with Medicago CCR1 or CCR2, harvested at 25 days post germination. In all cases error bars represent the SD of three replicates.

FIG. 10A-F: Overexpression of Medicago CCR1 and CCR2 in wild type Arabidopsis ecotype Col-0. FIG. 10A, RT-PCR screening to show expression of the Medicago transgenes in the Col-0 background. FIG. 10B, Visible appearance of plants at 20 days post-germination. FIG. 10C, Extractable activities of CCR measured with feruloyl CoA and caffeoyl CoA in stem extracts from wild type Arabidopsis (Col-0), and in Col-0 expressing Medicago CCR1 or CCR2. Results are expressed as a percentage of the average of that of Col-0. FIG. 10D, UV autofluorescence (panels a-c), phloroglucoinol staining (panels d-f) and Maule staining (panels g-i) of stem cross sections of wild type Arabidopsis Col-0 (panels a,d,g), and Col-0 expressing CCR1 (panels b,e,h) or CCR2 (panels c,f,i). FIG. 10E, Acetyl bromide lignin levels in the middle and basal portions of stems of wild type Arabidopsis (Col-0), and in Col-0 expressing with Medicago CCR1 or CCR2. All plants were 25 days post-germination. FIG. 10F, Lignin thioacidolysis yields and monomer compositions in the middle and basal portions of stems of wild type Arabidopsis (Col-0), and in Col-0 expressing with Medicago CCR1 or CCR2. All plants were 25 days post-germination. In all cases, error bars represent the SD of three replicates.

FIG. 11: Extractable CCR2 activities in alfalfa lines down-regulated in CCoAOMT expression. The results are expressed relative to the average control value. Error bars represent the SD of three replicates.

FIG. 12A-E: Gene expression in M. truncatula CCoAOMT Tntl transposon insertion mutants. FIG. 12A, Schematic shows the positions of independent Tnt1 insertions in CCoAOMT. FIG. 12B, RT-PCR screening to show reduction in CCoAOMT transcript levels in two independent Tnt1 insertion lines. FIG. 12C, Results show a protein gel blot analysis showing reduction of CCoAOMT and COMT protein levels in two independent Tnt1 insertion lines. FIG. 12D, Relative expression of CCR1, CCR2 and COMT transcripts in ccoaomt Tnt1 insertion lines compared to wild-type, as determined by qRT-PCR. FIG. 12E, Relative CCR1 and CCR2 extractable activities in ccoaomt Tnt1 insertion lines expressed as a percentage of that of wild type. In each case error bars represent the SD of three replicates.

FIG. 13A-C: Gene expression in M. truncatula CCR2 Tntl transposon insertion mutants. FIG. 13A, Relative expression of CCR1 and CCoAOMT transcripts in CCR2 Tnt1 insertion mutants compared to wild-type. FIG. 13B, Protein gel blot analysis showing increased CCoAOMT protein levels in two independent CCR2 Tntl insertion lines compared to wild type. FIG. 13C, Extractable activity of CCoAOMT in two independent CCR2 Tnt1 insertion lines expressed as a percentage of that of wild type. In each case error bars represent the SD of three replicates.

FIG. 14: A model to explain the retention of S lignin in Medicago plants with reduced CCoAOMT activity. Flux into G and S lignin is presented as occurring on two semi-independent metabolons, one (for G lignin biosynthesis) anchored to the cytoplasmic side of the ER through associations with the C3H P450, the other (for S lignin biosynthesis) through associations with the F5H P450. When CCoAOMT is active, feruloyl CoA is converted to coniferaldehyde (marked by a star) by CCR1. This can be directly converted to coniferyl alcohol, the G monolignol. High concentrations of feruloyl CoA inhibit CCR2, ensuring that flux proceeds via CCR1. However, the coniferaldehyde produced by CCR1 can diffuse to the F5H component of the second complex and ultimately be converted to S monolignol. If CCoAOMT is compromised, caffeoyl CoA will build up and is efficiently channeled, by virtue of the positively cooperative kinetics of CCR2, into the second complex which preferentially leads to S monolignol formation. Some of the coniferaldehyde produced by this route might escape the channel and provide substrate for CAD, thereby allowing the maintenance of the low G lignin levels observed following down-regulation of CCoAOMT.

### DESCRIPTION OF ILLUSTRATIVE EMBODIMENTS

The invention overcomes the limitations of the prior art by providing novel methods and compositions for the modification of plant lignin content. A major stumbling block to the use of biomass for production fuels is access to cell wall components that store a large portion of the solar energy converted by the plant Likewise, plant cell wall components are difficult for animals to digest and thus are not able to be efficiently converted into animal mass for human food (e.g., in grazing livestock). Much effort has been focused on genetic modification of plants to improve digestibility and energy yield from cell wall components. However, modifying the expression of many genes in the lignin synthesis and deposition pathway often has little effect on lignin content and/or results in severe phenotypes that render the resulting plants unusable for commercial processes.

The studies provided herein surprisingly demonstrate that altering the expression of CCR2, such as the M. truncatula CCR2, is highly effective in changing the lignin content of plants. Plants which overexpress CCR2 display increased lignin content and biomass. Accordingly, such plants can be used in production of biomass for processing into renewable biofuels. In particular, biofuel feed stocks with high lignin content can be used in gasification processes for production of biogas.

Conversely, plants comprising a down-regulated CCR2 gene exhibit nearly normal growth but have a lower level of stem lignin content. Plants with reduced lignin content are also useful in certain biofuel production processes, such as ethanol production. Likewise, reduction in lignin levels directly impacts forage digestibility in a parallel manner to the effects on enzymatic saccharifciation (Reddy et al., 2005; Chen and Dixon, 2007). Thus, forage plants down-regulated in CCR2 would be expected to exhibit improved digestibility.

The methods described here overcome the previous inability to alter global cell wall composition in the plants. The provided transgenic plants comprise altered lignin levels that render them useful in the production of improved agricultural products could not previously have been realized.

### I. PLANT TRANSFORMATION CONSTRUCTS

In certain aspects the invention concerns vectors for plant transformation and/or expression. Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. Vectors may be used to express a gene coding sequence such as a CCR2 coding sequence or a RNA sequences such as sequence complementary to all or part of CCR2 gene sequence.

It is contemplated that utilization of cloning systems with large insert capacities will allow introduction of large DNA sequences comprising more than one selected gene. In accordance with the invention, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al., (1996).

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. The PAL2 promoter may in particular be useful with the invention (U.S. Pat. Appl. Pub. 2004/0049802, the entire disclosure of which is specifically incorporated herein by reference). In one embodiment of the invention, the native promoter of a lignin biosynthesis coding sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is contemplated that vectors for use in accordance with the present invention may be constructed to include an ocs enhancer element. This element was first identified as a 16 by palindromic enhancer from the octopine synthase (ocs) gene of Agrobacterium (Ellis et al., 1987), and is present in at least 10 other promoters (Bouchez et al., 1989). The use of an enhancer element, such as the ocs element and particularly multiple copies of the element, may act to increase the level of transcription from adjacent promoters when applied in the context of plant transformation.

It is envisioned that lignin biosynthesis coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of a lignin biosynthesis coding sequence is used. Alternatively, a heterologous 3′ end may enhance the expression of sense or antisense lignin biosynthesis coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms “selectable” or “screenable” markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an a-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228). The gene that encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### II. ANTISENSE AND RNAi CONSTRUCTS

Antisense and RNAi treatments represent one way of altering lignin biosynthesis activity in accordance with the invention (e.g., by down-regulation of CCR2 gene expression). In particular, constructs comprising a lignin biosynthesis coding sequence, including fragments thereof, in antisense orientation, or combinations of sense and antisense orientation, may be used to decrease or effectively eliminate the expression of a lignin biosynthesis gene in a plant and obtain an improvement in lignin profile as is described herein. Accordingly, this may be used to “knock-out” the function of a lignin biosynthesis coding sequence or homologous sequences thereof.

Techniques for RNAi are well known in the art and are described in, for example, Lehner et al., (2004) and Downward (2004). The technique is based on the fact that double stranded RNA is capable of directing the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the invention, such an oligonucleotide may comprise any unique portion of a nucleic acid sequence provided herein. In certain embodiments of the invention, such a sequence comprises at least 18, 30, 50, 75 or 100 or more contiguous nucleic acids of the nucleic acid sequence of a lignin biosynthesis gene, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that a preferred embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., ribozyme; see above) could be designed. Methods for selection and design of sequences that generate RNAi are well known in the art (e.g., Reynolds, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### III. METHODS FOR GENETIC TRANSFORMATION

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species, including biofuel crop species, may be stably transformed, and these cells developed into transgenic plants.

A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Similarly, Agrobacterium mediated transformation has also proven to be effective in switchgrass. Somleva et al., (2002) describe the creation of approximately 600 transgenic switchgrass plants carrying a bar gene and a uidA gene (beta-glucuronidase) under control of a maize ubiquitin promoter and rice actin promoter respectively. Both genes were expressed in the primary transformants and could be inherited and expressed in subsequent generations. Addition of 50 to 200 μM acetosyringone to the inoculation medium increased the frequency of transgenic switchgrass plants recovered.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D′Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al., 1994), peanut (Singsit et al., 1997), cotton (McCabe and Martinell, 1993), tomato (VanEck et al., 1995), and legumes in general (U.S. Pat. No. 5,563,055, specifically incorporated herein by reference in its entirety).

Richards et al., (2001) describe the creation of transgenic switchgrass plants using particle bombardment. Callus was bombarded with a plasmid carrying a sgfp (green fluorescent protein) gene and a bar (bialaphos and Basta tolerance) gene under control of a rice actin promoter and maize ubiquitin promoter respectively. Plants regenerated from bombarded callus were Basta tolerant and expressed GFP. These primary transformants were then crossed with non-transgenic control plants, and Basta tolerance was observed in progeny plants, demonstrating inheritance of the bar gene.

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

E. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. BACTOAGAR, GELRITE, and GELGRO are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al., (1975) and MS media (Murashige and Skoog, 1962).

### IV. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318).

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103.

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and 14C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a selected lignin biosynthesis coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants.

As used herein the term “progeny” denotes the offspring of any generation of a parent plant prepared in accordance with the instant invention, wherein the progeny comprises a selected DNA construct. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. PRODUCTION OF BIOFUEL FROM LIGNOCELLULOSIC BIOMASS

The overall processes for the production of biofuels from plant matter well known in the art. As an example ethanol production typically involves two steps: saccharification and fermentation. First, saccharification produces fermentable sugars from the cellulose and hemicellulose in the lignocellulosic biomass. Second, those sugars are then fermented to produce ethanol. Thorough, detailed discussion of additional methods and protocols for the production of ethanol from biomass are reviewed in Wyman (1999); Gong et al., (1999); Sun and Cheng, (2002); and Olsson and Hahn-Hagerdal (1996).

A. Pretreatment

Raw biomass is typically pretreated to increase porosity, hydrolyze hemicellulose, remove lignin and reduce cellulose crystallinity, all in order to improve recovery of fermentable sugars from the cellulose polymer. As a preliminary step in pretreatment, the lignocellulosic material may be chipped or ground. The size of the biomass particles after chipping or grinding is typically between 0.2 and 30 mm. After chipping a number of other pretreatment options may be used to further prepare the biomass for saccharification and fermentation, including steam explosion, ammonia fiber explosion, acid hydrolysis.

1. Steam Explosion

Steam explosion is a very common method for pretreatment of lignocellulosic biomass and increases the amount of cellulose available for enzymatic hydrolysis (U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process causes hemicellulose degradation and lignin transformation. Addition of H₂SO₄, SO₂, or CO₂ to the steam explosion reaction can improve subsequent cellulose hydrolysis, decrease production of inhibitory compounds and lead to the more complete removal of hemicellulose (Morjanoff and Gray, 1987).

2. Ammonia Fiber Explosion (AFEX)

In AFEX pretreatment, the biomass is treated with approximately 1-2 kg ammonia per kg dry biomass for approximately 30 minutes at pressures of 1.5 to 2 MPa. (U.S. Pat. No. 4,600,590; U.S. Pat. No. 5,037,663; Mes-Hartree, et al., 1988). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002).

3. Acid Hydrolysis

Concentrated or dilute acids may also be used for pretreatment of lignocellulosic biomass. H₂SO₄ and HCl have been used at high, >70%, concentrations. In addition to pretreatment, concentrated acid may also be used for hydrolysis of cellulose (U.S. Pat. No. 5,972,118). Dilute acids can be used at either high (>160° C.) or low (<160° C.) temperatures, although high temperature is preferred for cellulose hydrolysis (Sun and Cheng, 2002). H₂SO₄ and HCl at concentrations of 0.3 to 2% (w/w) and treatment times ranging from minutes to 2 hours or longer can be used for dilute acid pretreatment.

Other pretreatments include alkaline hydrolysis, oxidative delignification, organosolv process, or biological pretreatment; see Sun and Cheng (2002).

B. Saccharification

After pretreatment, the cellulose in the lignocellulosic biomass may be hydrolyzed with cellulase enzymes. Cellulase catalyzes the breakdown of cellulose to release glucose which can then be fermented into ethanol.

Bacteria and fungi produce cellulases suitable for use in ethanol production (Duff and Murray, 1995). For example, Cellulomonas fimi and Thermomonospora fusca have been extensively studied for cellulase production. Among fungi, members of the Trichoderma genus, and in particular Trichoderma reesi, have been the most extensively studied. Numerous cellulases are available from commercial sources as well. Cellulases are usually actually a mixture of several different specific activities. First, endoglucanases create free chain ends of the cellulose fiber. Exoglucanases remove cellobiose units from the free chain ends and beta-glucosidase hydrolyzes cellobiose to produce free glucose.

Reaction conditions for enzymatic hydrolysis are typically around pH 4.8 at a temperature between 45 and 50° C. with incubations of between 10 and 120 hours. Cellulase loading can vary from around 5 to 35 filter paper units (FPU) of activity per gram of substrate Surfactants like Tween 20, 80, polyoxyethylene glycol or Tween 81 may also be used during enzyme hydrolysis to improve cellulose conversion. Additionally, combinations or mixtures of available cellulases and other enzymes may also lead to increased saccharification.

Aside from enzymatic hydrolysis, cellulose may also be hydrolyzed with weak acids or hydrochloric acid (Lee et al., 1999).

C. Fermentation

Once fermentable sugars have been produced from the lignocellulosic biomass, those sugars may be used to produce ethanol via fermentation. Fermentation processes for producing ethanol from lignocellulosic biomass are extensively reviewed in Olsson and Hahn-Hagerdal (1996). Briefly, for maximum efficiencies, both pentose sugars from the hemicellulose fraction of the lignocellulosic material (e.g., xylose) and hexose sugars from the cellulose fraction (e.g., glucose) should be utilized. Saccharomyces cerevisiae are widely used for fermentation of hexose sugars. Pentose sugars, released from the hemicellulose portion of the biomass, may be fermented using genetically engineered bacteria, including Escherichia coli (U.S. Pat. No. 5,000,000) or Zymomonas mobilis (Zhang et al., 1995). Fermentation with yeast strains is typically optimal around temperatures of 30 to 37° C.

D. Simultaneous Saccharification and Fermentation (SSF)

Cellulase activity is inhibited by its end products, cellobiose and glucose. Consequently, as saccharification proceeds, the build up of those end products increasingly inhibits continued hydrolysis of the cellulose substrate. Thus, the fermentation of sugars as they are produced in the saccharification process leads to improved efficiencies for cellulose utilization (e.g., U.S. Pat. No. 3,990,944). This process is known as simultaneous saccharification and fermentation (SSF), and is an alternative to the above described separate saccharification and fermentation steps. In addition to increased cellulose utilization, SSF also eliminates the need for a separate vessel and processing step. The optimal temperature for SSF is around 38° C., which is a compromise between the optimal temperatures of cellulose hydrolysis and sugar fermentation. SSF reactions can proceed up to 5 to 7 days.

E. Distillation

The final step for production of ethanol is distillation. The fermentation or SSF product is distilled using conventional methods producing ethanol, for instance 95% ethanol.

### VII. DEFINITIONS

Biofuel crop species: A plant that may be used to provide biomass for production of lignocellulosic-derived ethanol. Examples of such plants include switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass, and poplar, among others.

CCR2 coding sequence: As used herein a CCR2 coding sequence refers to a nucleic acid sequence encoding a functional cinnamoyl CoA reductase enzyme that exhibits greater enzymatic activity on caffeoyl CoA or 4-coumaroyl CoA substrates as compared to feruloyl CoA or sinapoyl CoA substrates.

Expression: The combination of intracellular processes, including transcription and translation undergone by a coding DNA molecule such as a structural gene to produce a polypeptide.

Forage crops: Crops including grasses and legumes used as fodder or silage for livestock production.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Phenotypes of Transgenic Alfalfa with Reduced Expression of Both COMT and CCoAOMT

Generation of transgenic alfalfa lines harboring RNAi constructs for the down-regulation of COMT or CCoAOMT has been previously reported (see, e.g., Chen et al., 2006). As observed in earlier studies using antisense constructs (Guo et al., 2001), RNAi-mediated down-regulation of CCoAOMT directed by the vascular-specific bean phenylalanine ammonia-lyse (PAL) 2 promoter results in reduced lignin yields with little if any effect on S lignin levels, whereas down-regulation of COMT drastically reduces S lignin (Chen et al., 2006). Down-regulation of either enzyme alone has little negative effect on plant growth.

If COMT and CCoAOMT are the only enzymes involved in monolignol O-methylation in alfalfa, and serve redundant functions, down-regulation of both enzymes together should lead to a major disruption of the lignin pathway. To study the effects of simultaneous down-regulation of both CCoAOMT and COMT, a cross was made between CCoAOMT-RNAi event Z2 used as the pollen source and COMT-RNAi event X3 (Chen et al., 2006). Both events were F1 progeny selected from a field evaluation for reduced lignin, increased digestibility and agronomic performance, and had previously been shown to possess single copy inserts based on F1 segregation PCR data. Progeny of the present cross were sorted by PCR to determine lines harboring single transgenes, both transgenes, or nulls. Three independent null, CCoAOMT alone and COMT alone progeny, and eight progeny harboring both CCoAOMT and COMT RNAi constructs (double), were selected and grown in a growth chamber. The double knock-down lines exhibited stunted phenotypes and significantly delayed time to flowering (Table 1). In fact, four lines (#s 8, 30, 31, and 41) grew so slowly that it took them over a year to attain seven internodes and these lines were not used for further analyses.

Stem samples (7 internodes) were harvested at the early flower bud stage, and protein extracts prepared for assay of CCoAOMT and COMT enzyme activities. Briefly, alfalfa stems (internodes 1 to 7) were collected and homogenized in liquid nitrogen. Powdered tissue (approximately 300 mg) was extracted for 1 h at 4° C. in extraction buffer (100 mM Tris-HCl, pH 7.5, 10% glycerol, 2 mM DTT, 0.2 mM MgCl₂, 1 mM phenylmethylsulfonyl fluoride) and the extracts desalted on disposable PD-10 columns (Amersham Biosciences). Protein concentrations were determined using Bradford dye binding reagent (Bio-Rad Laboratories, Inc.) with BSA as standard. The assay mixtures contained 5 μL of [¹⁴CH₃]-S-adenosyl-L-Met (0.6 mM, 13 μCi/μmol), 5 μL of caffeic acid or caffeoyl CoA (1 mM), 30 μL of assay buffer (100 mM Tris-HCl, pH 7.5, 10% glycerol, 2 mM DTT, 0.2 mM MgCl₂), and 10 μL of protein extract. They were incubated at 30° C. for 30 min, and stopped by adding 40 μL of 1 N HCl (for COMT assays) or 10 μL of 3 M NaOH followed by incubation at 37° C. for 10 min and acidification by adding 40 μL of 1 N HCl (for CCoAOMT assays). Labeled ferulic acid was extracted into 200 μL of hexane:ethyl acetate (1:1, v/v), and 150 μL of the separated organic layers were transferred to scintillation vials for determination of radioactivity.

Extractable CCoAOMT activity was unaffected in the COMT RNAi progeny, but was reduced to between 5-10% of null segregant activity in the CCoAOMT RNAi or CCoAOMT/COMT double RNAi lines. Interestingly, extractable COMT activity was higher in CCoAOMT knock-downs than in two out of three nulls, and was also higher in double knock-downs than in COMT single knockdowns, suggesting that down-regulation of CCoAOMT may up-regulate COMT expression (Table 1).

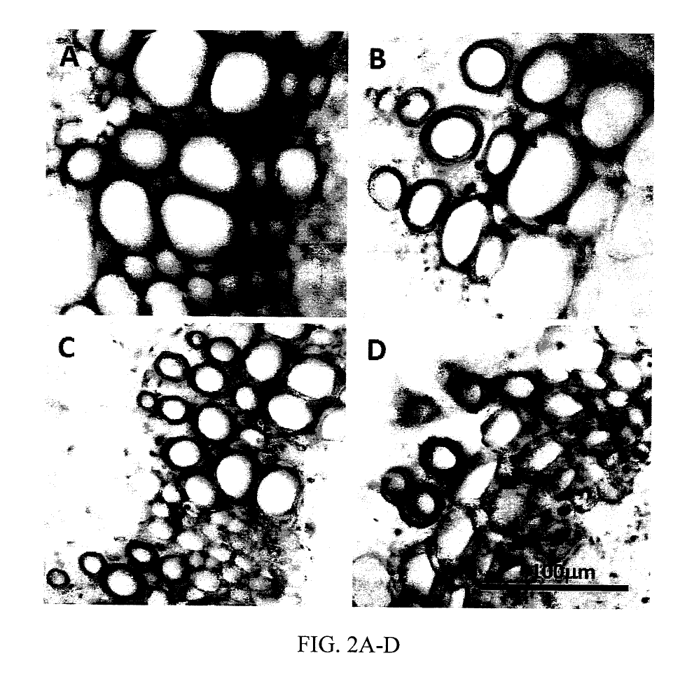

Examination of cross sections of the sixth internodes of transgenic plants by Maule staining and microscopy revealed the expected reduction in red staining (reflecting loss of S lignin) in lines down-regulated in COMT (FIG. 2A,B). In the null controls, S-lignin is intensely located in secondary walls of vascular tissues and pith rays (red-purple color) whereas COMT down-regulated lines did not show this coloration, indicating severely reduced amounts of S-lignin. The CCoAOMT down-regulated lines showed comparable amounts of S-lignin to the control (FIG. 2A,C), but COMT/CCoAOMT double down-regulated lines exhibited only a few vascular elements showing the presence of S-lignin. Equally striking was the swollen, doughnut-like appearance of the cell walls in the double knock-downs (FIG. 2D), a phenotype not observed in transgenic alfalfa down-regulated at any of the other steps in the monolignol pathway (Nakashima et al., 2008).

TABLE 1
COMT and CCoAOMT enzyme activities in internodes 1-7 of
control (null) alfalfa or transgenic plants down-regulated
in expression of COMT, CCoAOMT, or both (double).

| | COMT | CCoAOMT | Assigned | Days to |
|---|---|---|---|---|
| Plant # | activity* | activity* | genotype | harvest |
| 16 | 98 | 683 | Null | 75 |
| 48 | 137 | 1119 | Null | 75 |
| 69 | 87 | 595 | Null | 75 |
| 10 | 9 | 677 | COMT | 95 |
| 26 | 9 | 586 | COMT | 95 |
| 76 | 11 | 699 | COMT | 95 |
| 5 | 129 | 72 | CCoAOMT | 95 |
| 37 | 126 | 61 | CCoAOMT | 95 |
| 51 | 136 | 67 | CCoAOMT | 95 |
| 3 | 26 | 35 | Double | 130 |
| 22 | 25 | 37 | Double | 130 |
| 25 | 29 | 59 | Double | 130 |
| 56 | 18 | 30 | Double | 104 |
| 63 | 21 | 27 | Double | 104 |
| 67 | 38 | 62 | Double | 104 |
| 73 | 16 | 25 | Double | 104 |

*Activity is expressed a ¹⁴C-dpm/μg protein.

### Example 2

### The Lignin Phenotype of Double OMT Knock-Downs Suggests a Role for COMT in the 3-O-Methylation of G Lignin Precursors

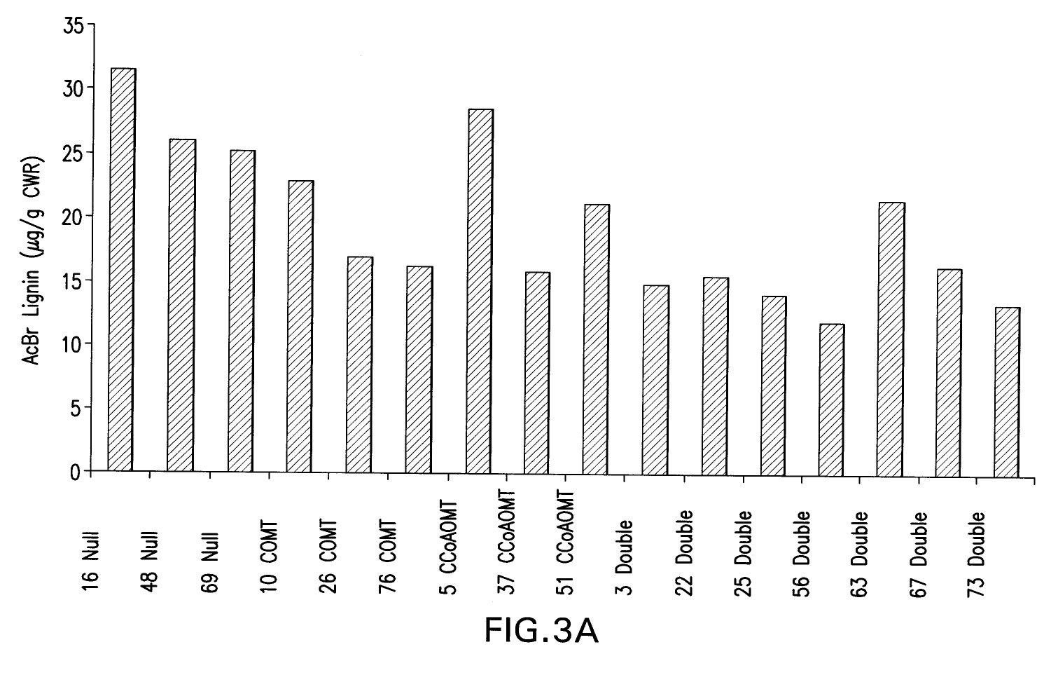

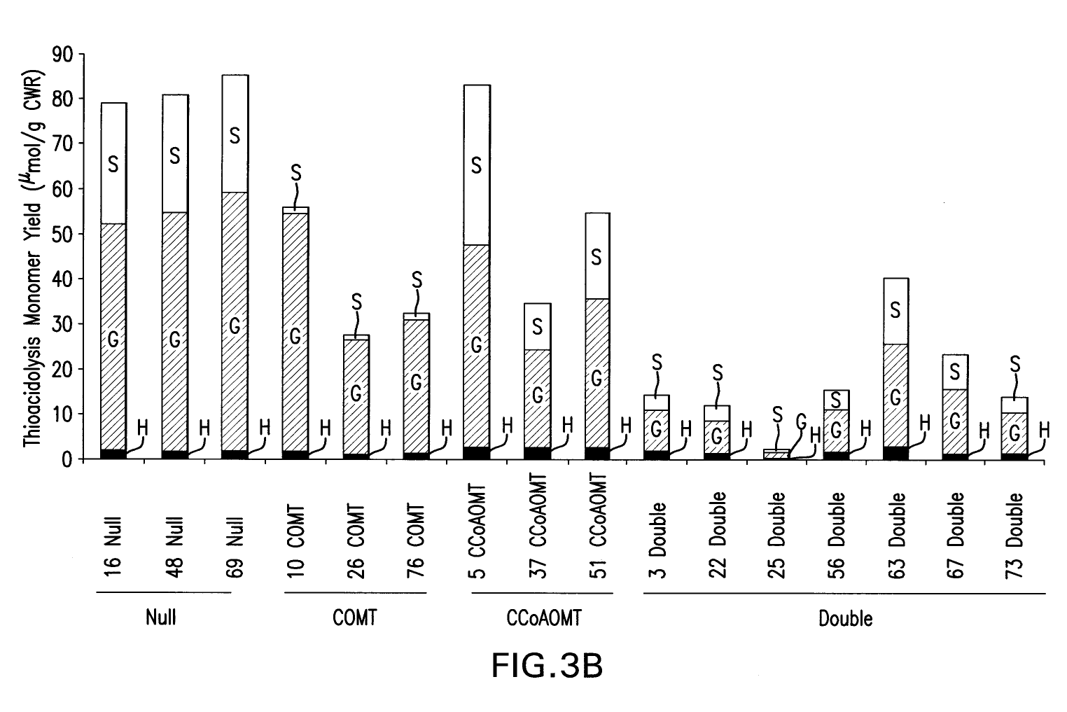

To determine lignin content and composition in the various progeny lines, stem samples were analyzed by the acetyl bromide method for total lignin content, and by thioacidolysis to determine monomer yield and composition (FIG. 3). Acetyl bromide lignin levels were reduced in all COMT and CCoAOMT single RNAi progeny when compared to the nulls, with the exception of CCoAOMT line 5, but were reduced further in some of the double knock-downs (FIG. 3A). The reduction in lignin in the double knock-downs was even more apparent when considered in terms of total thioacidolysis yield (FIG. 3B). However, six of the seven double knock-down lines, although exhibiting reduced S-lignin levels, produced more S-lignin than did any of the single COMT RNAi lines (FIG. 3B), and the S/G ratios of the double RNAi lines were, on average, only slightly lower than those of the nulls. The relative proportion of H to total monomer units was greater in the double knock-downs, suggesting that they were less impaired in H lignin synthesis than in G and S lignin synthesis.

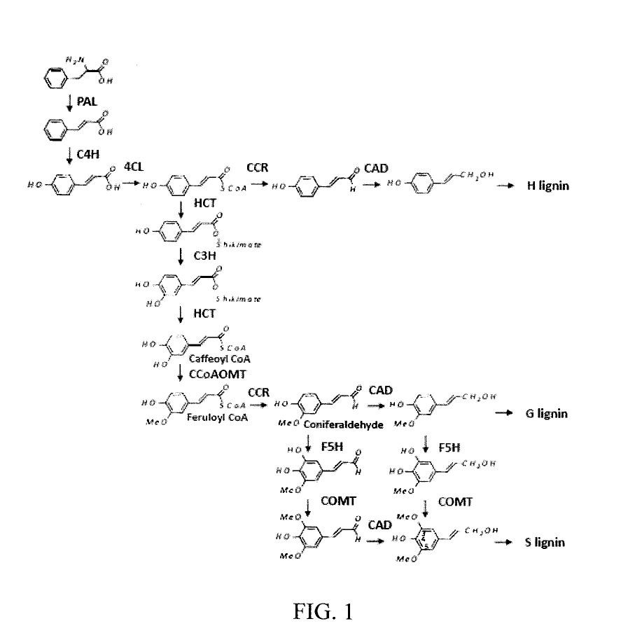

Down-regulation of COMT alone almost completely eliminates S-lignin, and has a similar effect on G-lignin as down-regulation of CCoAOMT, which itself has little effect on S lignin levels. This, coupled with the apparently additive effect of down-regulating both COMT and CCoAOMT on lignin quantity, is consistent with a model in which COMT alone acts to methylate the 5-position of the 5-hydroxyguaiacyl unit in the synthesis of syringyl monomers (FIG. 1), but also acts in addition to CCoAOMT as a 3-O-methyltransferase in the formation of G monomers. These studies are also consistent with COMT being the primary enzyme for introducing the 3-O-methyl group into precursors destined for the syringyl lignin pathway.

### Example 3

### Substrate Preferences of Medicago CCRs Suggest the Involvement of Different Enzymes for Reduction of Caffeoyl and Feruloyl CoAs

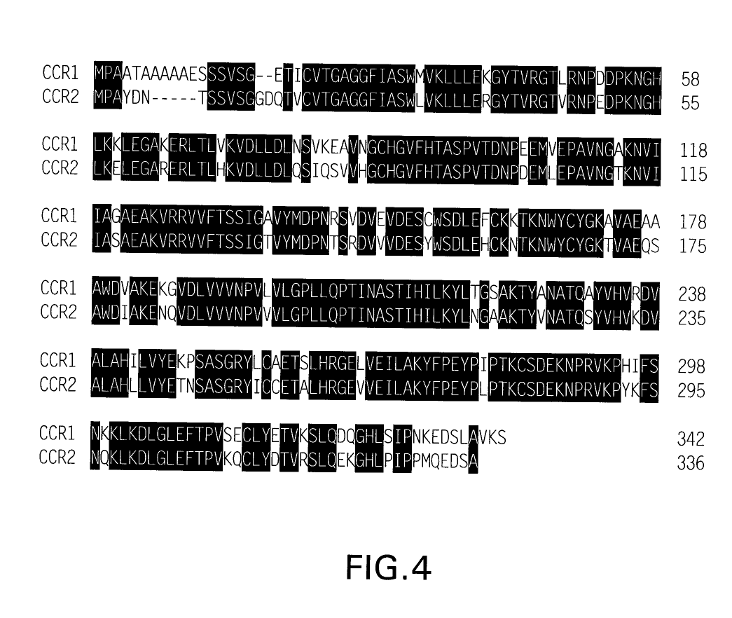

Based on the conventional understanding of monolignol biosynthesis, 3-O-methylation by COMT would most likely occur at the level of caffeoyl aldehyde or caffeoyl alcohol, preferred substrates for the enzyme from alfalfa (Parvathi et al., 2001). This raises the question of the origin of caffeoyl aldehyde. Caffeoyl CoA has been described as a poor substrate for cinnamoyl CoA reductases (CCRs) from plants (Li et al., 2001; Patten et al., 2005), although this activity is present in crude extracts from alfalfa (Guo et al., 2002). The model legume M. truncatula is a species very closely related to alfalfa, but, in contrast to alfalfa, has a near complete genome sequence and excellent genetic resources. For this reason, the M. truncatula model was chosen to address the substrate preferences of CCR using the model organism. M. truncatula possess at least eight CCR and CCR-like genes, two of which (CCR1 and CCR2, corresponding to TC #s 106830 and 100678 respectively in the Medicago gene index available on the internet at compbio.dfci.harvard.edu/tgi/cgibin/tgi/gimain.pl?gudb=Medicago) cluster phylogenetically with CCRs previously shown to function in lignin biosynthesis (Jackson et al., 2008). An amino acid sequence alignment of CCR1 and CCR2 is shown in FIG. 4. The two proteins share an overall sequence identity of 80.2% and both contain the motif KNWYCYGK (SEQ ID NO: 72), which is highly conserved in functional CCR enzymes and is believed to be critical for catalysis (Lacombe et al., 1997).

To assess the potential of Medicago CCRs to produce caffeoyl aldehyde, CCR1 and CCR2 were expressed in E. coli and assayed the purified recombinant enzymes with varying concentrations of each of the four potential substrates, 4-coumaroyl-, caffeoyl-, feruloyl- and sinapoyl-CoAs. Briefly, the coding regions of CCR1 and CCR2 were amplified from the original cDNA clones with introduction of NheI enzyme sites before the ATG start codons, a BamHI site after the CCR1 stop codon, and an XhoI site after the CCR2 stop codon. The primers EpCCR1F and EpCCR1R (see Table 3 below) were used for amplification of CCR1, and EpCCR2F and EpCCR2R were used for amplification of CCR2. The PCR products were cloned into T-vector (Promega). After confirmation by sequencing, both CCR sequences were subcloned into pET28a vector (Novagen) and the resulting constructs introduced into E. coli strain BL21 (DE34) (Novagen). The clones with correct inserts were grown at 37° C. until OD₆₀₀ reached about 0.6, IPTG was added to a final concentration at 0.25 mM, and culture continued at 16° C. overnight. Induced E. coli cells were harvested by centrifugation at 5,000 g for 10 min, frozen in liquid nitrogen and stored at −80° C.

All enzyme purification steps were carried out at 4° C. The harvested cells were suspended in extraction buffer (100 mM Hepes-NaOH, pH 7.5, 500 mM NaCl, 10 mM imidazole, 10% glycerol, 10 mM mercaptoethanol, 1 mM EDTA, and 1 mM PMSF), sonicated three times for 20 s and centrifuged at 16,000 g for 10 min. The supernatants were mixed with Ni-NTA His-bind resin (Promega). After washing three times in extraction buffer without PMSF, the recombinant proteins were eluted with elution buffer (100 mM Hepes-NaOH, 10% glycerol, 1 mM EDTA, 10 mM mercaptoethanol, and 500 mM imidazole) and the protein stored at −80 ° C. until assay.

For CCR enzyme activity assays purified CCR protein (2.5 μg) was mixed with assay buffer (100 mM phosphate pH 6.25, 10 mM 2-mercaptoethanol, 0.2 mM NADPH and cinnamoyl CoA substrates at the indicated concentration) in a final volume of 500 μl. The reaction was carried out at 30° C. for 5 min and then terminated with the addition of 70 μL 24% trichloroacetic acid. The reaction mixture was extracted with ethyl acetate (0.6 ml×3) and the combined organic phases dried under a stream of N₂. The pellet was re-suspended in 50 μL methanol and 25 μL was subjected to HPLC analysis out on a Beckman System Gold HPLC system consisting of a programmable solvent module 126, a System Gold 508 autosampler and a System Gold 168 diode array detector. A Waters Spherisorb ODS-2 5 μm reverse phase column (5 μm particle, 250×4.6 mm) was used. Compounds were identified by comparing their UV spectra and retention times with authentic standards of hydroxycinnamoyl aldehydes, and quantified by means of standard curves with correction for recovery of internal standards. Protein concentrations were determined using Bradford reagent (Bio-Rad). For kinetic analysis at various substrate concentrations, enzyme velocity curves were analyzed using Sigmaplot 10 software (Systat Software Inc.).

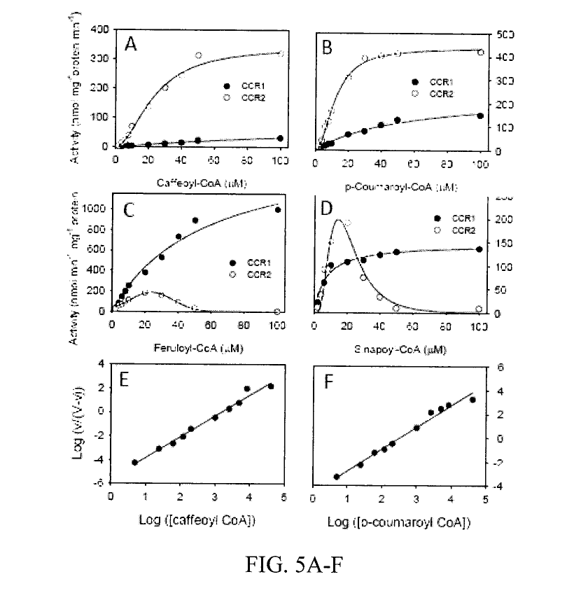

Results for CCR1 showed the higher turnover number, and was most active with feruloyl CoA (FIG. 5C). However, it exhibited very low activity with caffeoyl CoA (FIG. 5A). In contrast, CCR2 was much more active than CCR1 with caffeoyl and 4-coumaroyl CoAs (FIG. 5A,B), and its activity with both feruloyl and sinapoyl CoAs was strongly inhibited at higher substrate concentrations (FIG. 5C,D). Curve fitting with Sigmaplot 10 software suggested a sigmoidal response of CCR2 activity to increasing coumaroyl and caffeoyl CoA concentrations, and further kinetic analysis confirmed that CCR2 exhibits positive cooperativity with caffeoyl and 4-coumaroylCoAs, with a Hill coefficient of 1.9-2.0 (FIG. 5E,F). Interestingly, CCR2 was almost twice as active as CCR1 at 20 μM sinapoyl CoA, but had virtually no activity at 50 μM, a concentration at which CCR1 activity was saturated. The kinetic constants for CCR1 and CCR2 are shown in Table 2. Based on the calculated catalytic efficiency (Kcat/Km), feruloyl CoA was the preferred substrate for CCR1, consistent with reports on CCRs from eucalyptus (Goffner et al., 1994), poplar (Li et al., 2005), and wheat (Ma, 2007). Likewise, caffeoyl and coumaroyl CoAs were the preferred substrates for CCR2, at least in vitro.

The other CCR-like genes from Medicago (Jackson et al., 2008) were also expressed in E. coli and the recombinant proteins tested for activity with hydroxycinnamoyl CoA substrates; none was found to be active.

TABLE 2
Kinetic constants for Medicago CCR1 and CCR2. Data represent mean values from three replicate sets of
assays. S_{0.5} values (substrate concentration giving half maximum velocity) are given in place of Km values for CCR2 with coumaroyl
and caffeoyl CoAs in view of the sigmodial velocity curves.

| | Km/S_{0.5} (μM) | | Vmax (μmol min⁻¹) | | Kcat (min⁻¹) | | Kcat/Km (μM⁻¹ min⁻¹) | |
|---|---|---|---|---|---|---|---|---|
| | CCR1 | CCR2 | CCR1 | CCR2 | CCR1 | CCR2 | CCR1 | CCR2 |
| Feruloyl CoA | 54.5 ± 3.2 | 44.5 ± 4.5 | 1.64 ± 0.11 | 0.48 ± 0.05 | 60.1 ± 2.05 | 18.1 ± 2.0 | 1.14 ± 0.08 | 0.40 ± 0.05 |
| Sinapoyl CoA | 7.2 ± 0.4 | 32.7 ± 3.6 | 0.15 ± 0.01 | 0.32 ± 0.02 | 5.5 ± 0.14 | 12.0 ± 1.1 | 0.68 ± 0.05 | 0.37 ± 0.05 |
| Caffeoyl CoA | 161 ± 6.8 | 23.4 ± 0.80 | 0.085 ± 0.0 | 0.35 ± 0.01 | 3.1 ± 0.07 | 9.0 ± 0.3 | 0.019 ± 0.0 | 0.49 ± 0.02 |
| Coumaroyl CoA | 56.8 ± 4.7 | 12.4 ± 1.0 | 0.25 ± 0.01 | 0.44 ± 0.01 | 9.0 ± 0.10 | 17.4 ± 0.4 | 0.16 ± 0.1 | 0.90 ± 0.03 |

### Example 4

### Tissue-Specific Expression of CCR1 and CCR2

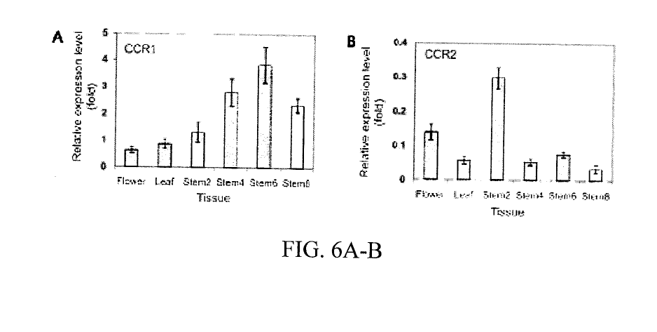

Quantitative real time PCR was used to determine CCR1 and CCR2 transcript levels in various tissues of M. truncatula (see FIG. 6A-B). Both genes were expressed in stems, leaves and flowers; CCR1 was expressed overall at about 10-times the level of CCR2, and was most highly expressed in the sixth internode of the stem, whereas CCR2 was most highly expressed in the less mature second internode. A more detailed expression analysis through mining the data in the Medicago Gene Expression Atlas (Benedito et al., 2008) indicated that both genes were most highly expressed in roots. However, expression of CCR1 decreased following nodulation of roots, whereas expression of CCR2 was higher in nodulated than in non-nodulated roots, potentially suggesting non-redundant functions in roots.

In situ hybridization of cross sections of the second internodes of Medicago stems revealed that CCR1 is expressed in the vascular elements, with weaker expression in the interfascicular (xylem fiber) region. CCR2 and CCoAOMT exhibited a similar expression pattern, although the ratio of expression in vascular elements compared to interfascicular tissue was greater.

### Example 5

### Genetic Loss-of-Function Analysis of CCR1 and CCR2

To address whether the different in vitro substrate preferences of CCR1 and CCR2 have functional consequences for lignin biosynthesis in Medicago, knock-out mutations in these genes were generated in M. truncatula. A series of PCR primers from the open reading frame sequences of CCR1 and CCR2 (see Table 3) were used to amplify DNA pools from a large population of M. truncatula lines harboring multiple copies of the tobacco Tnt1 retrotransposon (Tadege et al., 2008). This resulted in the identification of two lines with transposon inserts in CCR1, and three lines with inserts in CCR2.

TABLE 3
DNA primers used in the present studies.

| Primer name | Primer sequence (5′->3′) |
|---|---|
| Actin-F | AGTAACTGGGATGACATGGA (SEQ ID NO: 1) |
| Actin-R | TAACCCTCATAGATTGGCAC (SEQ ID NO: 2) |
| AtActin-F | TGACAATGGGACAGGAATGGT (SEQ ID NO: 3) |
| AtActin-R | CAGCCCTTGGAGCATCATCT (SEQ ID NO: 4) |
| CCoAOMT-F | TCACCCACATCCAACCGTCCATCT (SEQ ID NO: 5) |
| CCoAOMT-R | TGCTTAATTTGTAGCTCCCACACA (SEQ ID NO: 6) |
| CCR1-F | ACAAAGTAACGTCACACCAACT (SEQ ID NO: 7) |
| CCR1-R | ACAATGCATGGGGTCTATTCATAAC (SEQ ID NO: 8) |
| CCR2-F | TGCCATATTGCTCAATGTGTCACT (SEQ ID NO: 9) |
| CCR2-R | TCTTAAAACCCCTCTATAAGAGTAC (SEQ ID NO: 10) |
| EpCCR1-F | CGCTAGCATGCCTGCCGCTACCG (SEQ ID NO: 11) |
| EpCCR1-R | CGGATCCTTAGGATTTGACTGCTAGAGAATC (SEQ ID NO: 12) |
| EpCCR2-F | CGCTAGCATGCCTGCCTATGATAACACTTC (SEQ ID NO: 13) |
| EpCCR2-R | GCTCGAGTTAAGCAGAGTCTTCTTGCATGG (SEQ ID NO: 14) |
| IS-CCoAOMT-F | AGAAGGTCCAGCTCTTCCAGTTCT (SEQ ID NO: 15) |
| IS-CCoAOMT-T7-F | GCGTAATACGACTCACTATAGGGAGAAGGTCCAGCTCTTCCAGTTCT (SEQ ID NO: 16) |
| IS-CCoAOMT-R | ACTAACCAATGCAATTGGCTTCAAA (SEQ ID NO: 17) |
| IS-CCoAOMT-T7-R | GCGTAATACGACTCACTATAGGGACTAACCAATGCAATTGGCTTCAAA (SEQ ID NO: 18) |
| IS-CCR1-F | GGGATTGGAATTTACACCAGTGAG (SEQ ID NO: 19) |
| IS-CCR1-T7-F | GCGTAATACGACTCACTATAGGGGGGATTGGAATTTACACCAGTGAG (SEQ ID NO: 20) |
| IS-CCR1-R | GGCAAATAATTGAGTTGAATGCTCAAG (SEQ ID NO: 21) |
| IS-CCR1-T7-R | GCGTAATACGACTCACTATAGGGGGCAAATAATTGAGTTGAATGCTCAAG (SEQ ID NO: 22) |
| IS-CCR2-F | ATCAAAAGCTGAAAGACTTG (SEQ ID NO: 23) |
| IS-CCR2-T7-F | GCGTAATACGACTCACTATAGGGATCAAAAGCTGAAAGACTTG (SEQ ID NO: 24) |
| IS-CCR2-R | TTGGTACAATCTTTAAAACTAGT (SEQ ID NO: 25) |
| IS-CCR2-T7-R | GCGTAATACGACTCACTATAGGGTTGGTACAATCTTTAAAACTAGT (SEQ ID NO: 26) |
| qCCoAOMT-F | GATCTTGTTAAAGTGGGAGGTGTGA (SEQ ID NO: 27) |
| qCCoAOMT-R | GTGCAACCACAGATCCATTCC (SEQ ID NO: 28) |
| qCCR1-F | AGGGATGTTGCATTAGCTCACA (SEQ ID NO: 29) |
| qCCR1-R | TCAGCACATAAGTATCTACCAGAAGCA (SEQ ID NO: 30) |
| qCCR2-F | GCGACCAAACCGTGTGTGT (SEQ ID NO: 31) |
| qCCR2-R | AAGAGAAGTTTGACAAGCCAAGAAG (SEQ ID NO: 32) |
| qCOMT-F | AAAGTGATTGTGGCAGAATGCA (SEQ ID NO: 33) |
| qCOMT-R | TTTTGTGGCCAGGCTTGAA (SEQ ID NO: 34) |
| Tnt1-F | TCCTTGTTGGATTGGTAGCCAACTTTGTTG (SEQ ID NO: 35) |
| Tnt1-R | CAGTGAACGAGCAGAACCTGTG (SEQ ID NO: 36) |

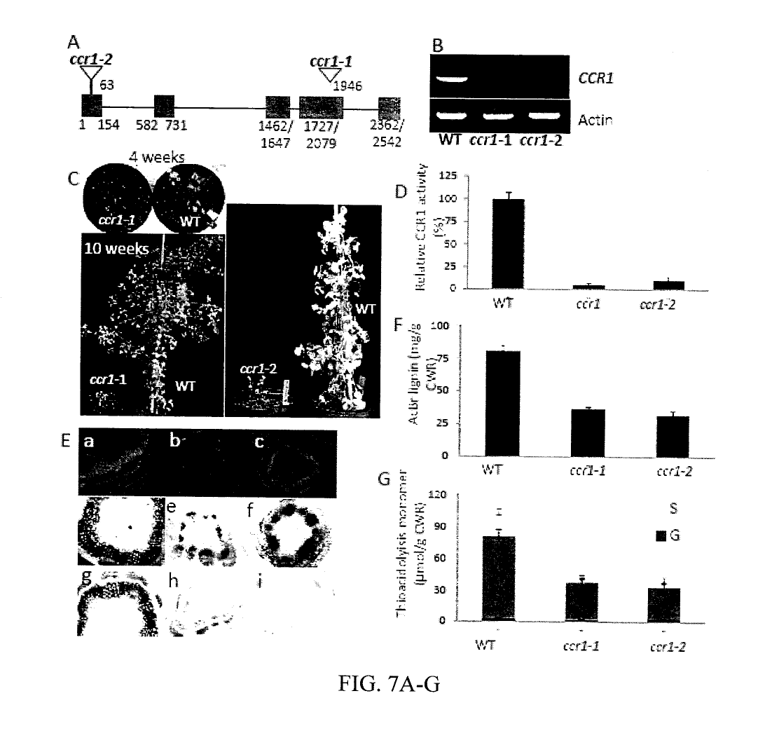

The two ccr1 mutant lines, NF4532 and NF5 145 (referred to as ccr1-1 and ccr1-2), had Tnt1-retrotransposon inserts in the fourth and first exons of the CCR1 gene, respectively (FIG. 7A), and this resulted in a severe reduction in CCR1 transcript levels in both lines as revealed by RT-PCR (FIG. 7B). ccr1-1 and ccr1-2 homozygotes exhibited very stunted growth and did not survive after flowering (FIG. 7C), whereas heterozygotes appeared to grow normally. As CCR1 prefers feruloyl CoA and CCR2 was inactive at high concentrations of this substrate (FIG. 5C), CCR1 activity alone could be measured in crude extracts with feruloyl CoA at 50 μM. Based on this assumption, activity assays indicated that CCR1 activity was strongly reduced in the ccr1 mutant, to less than 15% of that in the wild type (FIG. 7D).

Lignin levels were significantly reduced in the stems of the ccr1 knockout mutants, as demonstrated by reduced autofluoresence (FIG. 7E panels a-c) and lower intensity of phloroglucinol staining in the vascular tissue (FIG. 7E panels d-f) in comparison with that in the wild type. Maule staining revealed a dramatic decrease in S lignin levels in vascular tissue (FIG. 7E, panels h,i) compared to wild type (FIG. 7E, panel g). The decreases in total and S lignin were confirmed by extraction and chemical analysis. Acetyl bromide lignin was reduced by more than 50% in the ccr1 knockout mutants (FIG. 7F), and thioacidolysis revealed that the reduction in S monomer content was more than that of G monomers, leading to a reduction of S/G ratio to 0.15-0.19 in the ccr1 mutants compared to 0.29 in the wild type (FIG. 7G).

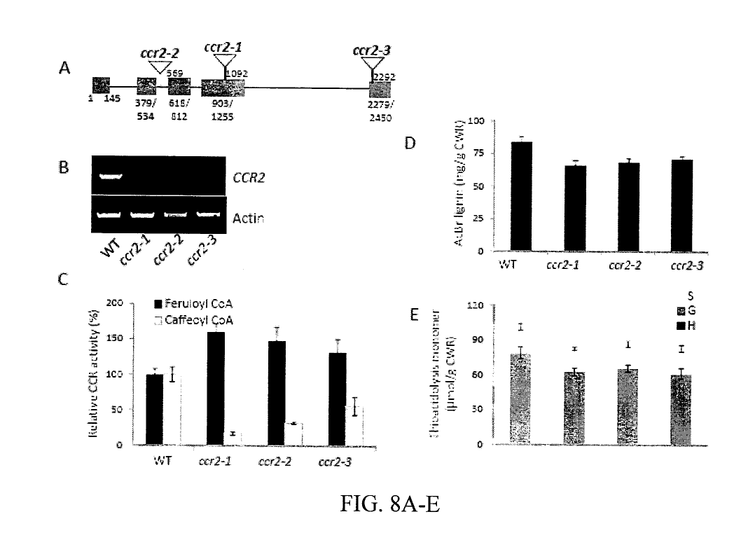

Three lines (NF4418, NF7205 and NF10441, referred to as ccr2-1, ccr2-2 and ccr2-3) had Tnt1 insertions in the CCR2 gene (FIG. 8A), and RT-PCR confirmed that no transcript corresponding to the size of the CCR2 mRNA was detectable in these lines (FIG. 8B). The mutants did not show any visible phenotypes under the growth condition. As CCR1 activity is only around 7% that of CCR2 activity at 50 μM caffeoyl CoA (FIG. 5A), the extractable activity measured under these conditions predominantly reflects CCR2 activity (FIG. 8C). Disruption of CCR2 expression led to a strong decrease in the extractable activity toward caffeoyl CoA, whereas the activity toward feruloyl CoA increased by 30-60% (FIG. 8C). The CCR2 knock-out lines exhibited an approximately 10% reduction in acetyl bromide lignin and an approximately 25% reduction in total thioacidolysis yield (FIG. 8D,E), with G lignin being more strongly reduced than S lignin, resulting in an increase in S/G ratio from 0.29 to 0.33 (FIG. 6E).

### Example 6

### Complementation of Arabidopsis ccr1 Mutants with Medicago CCR1 and CCR2

Two CCR genes have been identified in the Arabidopsis genome (Lauvergeat et al., 2001). Arabidopsis CCR1 and CCR2 enzymes both possess substrate preferences similar to that of Medicago CCR1 (Lauvergeat et al., 2001; Patten et al., 2005), and there has been no report of an Arabidopsis CCR with preference for caffeoyl CoA. To further address the potential functions of Medicago CCR1 and CCR2 in lignin biosynthesis, complementation experiments were undertaken in the Arabidopsis irx4 ccr1 mutant with each of the two Medicago genes under control of the constitutive 35S promoter. The irx4 mutation is in ecotype Landsberg erecta (Ler) with a point mutation in the highly conserved intron splice site sequence in the second intron of the CCR1 gene (Jones et al., 2001). Briefly, the ORFs of CCR1 and CCR2 were cloned into the Gateway Entry vector pENTR/DTOPO (Invitrogen), and confirmed by sequencing. For stable transformation by Agrobacterium tumefaciens, the Medicago CCR1 and CCR2 ORFs were transferred into the Gateway plant transformation destination vector pB2GW7 (Karimi et al., 2002) using Gateway LR Clonase enzyme mix with pENTR-CCR1 and CCR2 according to the manufacturer's instructions (Invitrogen). The reading frames of the resulting vectors, pB2GW7-CCR1 and pB2GW7-CCR2, were confirmed by sequencing, and the vectors transformed into A. tumefaciens strain AGL1 by electroporation. A single colony containing the target construct was confirmed by PCR and used for genetic transformation of Arabidopsis using the floral dip techniques (Clough and Bent, 1998). The transformants were selected on 10 mg/L PPT.

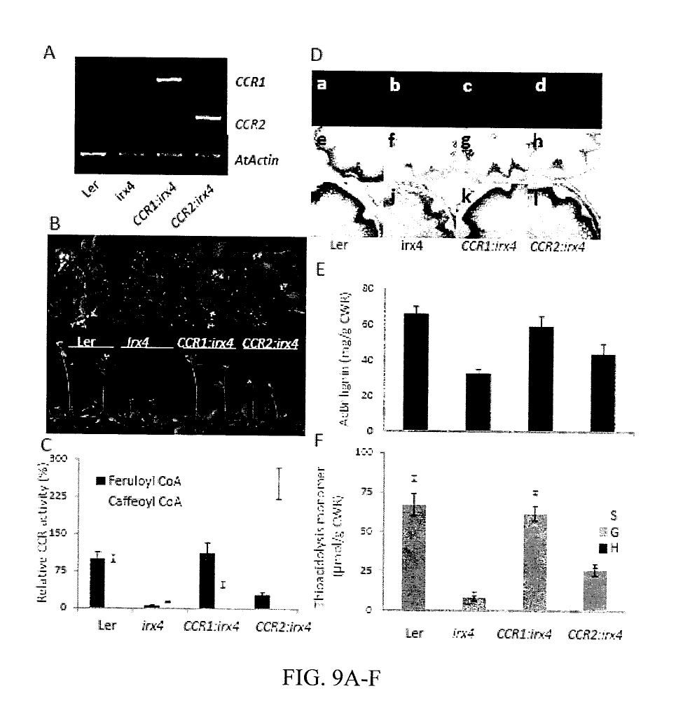

More than 20 independent transformants were obtained, and the expression of Medicago CCR1 or CCR2 was confirmed by RT-PCR (FIG. 9A). Two or three independent transformants with high expression of the Medicago genes were chosen for further analysis. The results obtained with these lines were similar and representative results from one line are presented here.

As reported previously, the irx4 mutant exhibited stunted growth (Jones et al., 2001). The rosette leaves of the mutant were much smaller and spoon-shaped when compared to those of Ler, and the inflorescence stem was shorter and weaker (FIG. 9B). The CCR activity toward feruloyl and caffeoyl CoAs was reduced by 94% and 87% of that in Ler, respectively (FIG. 9C). The total lignin level in the stem was also significantly reduced, based on autofluorescence and histochemical staining with phloroglucinol or Maule reagent (compare FIG. 9D, panels b,f,j with FIG. 9D, panels a,e,i), and by determination of acetyl bromide lignin level (FIG. 9E). Maule staining (FIG. 9D, panels i-j) and thioacidolysis (FIG. 9F) revealed a dramatic decrease in S monomers in the mutant compared to Ler, leading to a reduction in S/G ratio from 0.24 to 0.05 (FIG. 9F).

Expression of Medicago CCR1 in the irx4 background led to a complete recovery of visible phenotype (FIG. 9B). The extractable CCR activity toward feruloyl CoA increased by about 20-fold to a level comparable to that in Ler, and activity toward caffeoyl CoA increased by 3.6-fold relative to that in irx4, but only attained 48% of that in Ler (FIG. 9C). Total lignin production was restored (FIG. 9D, panels c,g,k; FIG. 9E,F), and the S/G ratio returned to a value of about 0.21 (FIG. 9F).

In contrast, Medicago CCR2 only partially complemented the irx4 phenotype; the leaf shape and color were more like these in wild type Ler, but plant growth, although increased, did not reach that of Ler (FIG. 9A). Expression of CCR2 led to a large increase in the extractable CCR activity toward caffeoyl CoA, by 17-fold compared to that in irx4 and 2.5 times that in Ler (FIG. 9C), but activity toward feruloyl CoA was only increased to about 30% of that in Ler (FIG. 9C). The decreases in total lignin, G and S monomers and S/G ratio in irx4 were all partially restored upon expression of CCR2 (FIG. 9D, panels d,h,j; FIG. 9E,F).

### Example 7

### Overexpression Medicago CCR1 and CCR2 in Wild Type Arabidopsis

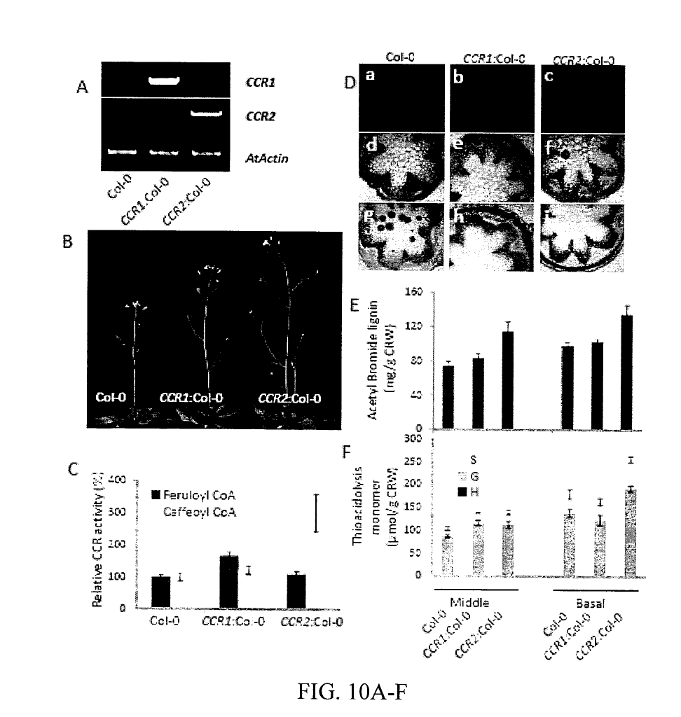

Because Medicago CCR2 can clearly function to (partially) restore lignin synthesis in Arabidopsis lacking CCR1 expression, it was next determined whether true over-expression of Medicago CCRs could impact the extent of lignification in Arabidopsis. Medicago CCR1 and CCR2 were expressed in wild type Arabidopsis ecotype Columbia-0 (Col-0) under control of the constitutive 35S promoter, and the expression of the transgenes confirmed by RT-PCR (FIG. 10A). Around 50 transformants for each gene were obtained and the transgenic lines overexpressing CCR1 exhibited a slightly faster growing inflorescence (FIG. 10B), and 67% and 10% increases in extractable CCR activity with feruloyl CoA and caffeoyl CoA, respectively (FIG. 10C); in contrast, overexpression of CCR2 resulted in slightly earlier bolting, and increased growth and branching of the inflorescence (FIG. 10B). Stems of plants expressing CCR2 had similar extractable CCR activity toward feruloyl CoA to Col-0, but a large increase (300%) in activity toward caffeoyl CoA (FIG. 10C).

For measurement of CCR activities in crude plant extracts described above, tissues were homogenized as described above for OMT assays. Caffeoyl CoA (50 μM) was used as substrate to estimate CCR2 activity, and feruloyl CoA (50 μM) was used for estimation of CCR1 activity. An aliquot of the crude extract (50 μL) was mixed with the CCR assay buffer and reactions incubated at 30° C. for 30 min. The products, coniferaldehyde or caffeoyl aldehyde, were quantified as previously indicated. As the crude extract contains cinnamyl alcohol dehydrogenase, which converts the cinnamyl aldehydes to the corresponding alcohols, the cinnamyl alcohols were also measured in the HPLC analysis and counted towards the CCR activity.

Because of the increase in the rate of inflorescence development in the Arabidopsis lines expressing Medicago CCR, lignin content and composition were analyzed in the basal, most mature part of the inflorescence stems of transgenic plants at 20 days after sowing, in order to minimize differences due to developmental stage. UV autofluorescence (FIG. 10D, panels a-c), phloroglucinol staining (FIG. 10D, panels d-f) and Maule staining (FIG. 10D, panels g-i) all indicated a small increase in lignin levels in the CCR1 over-expressing lines and a greater increase in the CCR2 expressing lines. The increased red coloration in the vascular tissue of the CCR2 expressing line (FIG. 10D, panel i) suggested a strong increase in S lignin. Overexpression of the Medicago genes did not change the spatial pattern of lignification (FIG. 10D).

To better characterize lignin accumulation during stem development in the transgenic plants, the inflorescences of 25 day-old plants were divided into three equal lengths. The upper part consisted of flowers and very young stem tissue; the lignin level was extremely low and these samples were not analyzed further. The middle part represented the developing stage and the basal part the mature stage. Acetyl bromide lignin levels in both the middle and lower segments were higher in the CCR2 expressing lines than in controls, whereas plants expressing CCR1 had similar acetyl bromide lignin levels in these tissues (FIG. 10E). Thioacidolysis revealed small increases in both G and S lignins in the middle portions of the stems of plants expressing either CCR1 or CCR2, whereas only the CCR2 expressors exhibited increased lignin levels in the basal parts (FIG. 10F).

### Example 8

### Cross-Talk between the CCoAOMT and CCR2 Pathways

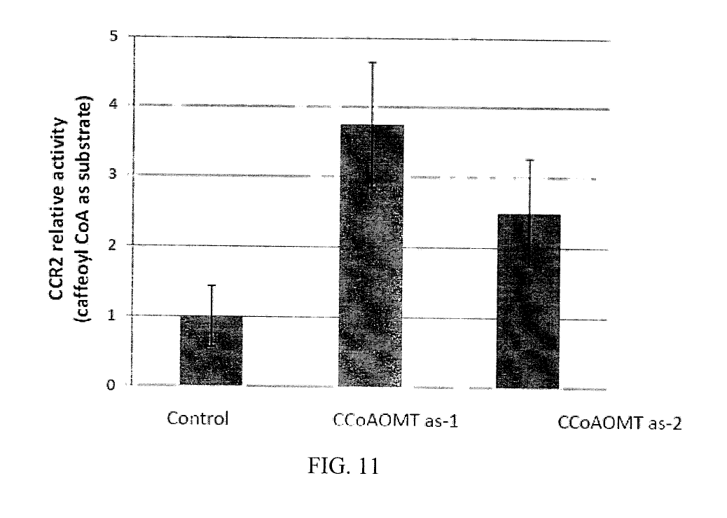

After establishing that Medicago possess a route, via CCR2 and COMT, that can operate in the 3-O-methylation methylation of monolignol precursors, it was nest determined how this might function to maintain S lignin levels in CCoAOMT down-regulated plants by examining the effects of CCoAOMT down-regulation on CCR2 and COMT expression. First, existing alfalfa lines in which CCoAOMT was down-regulated through antisense expression were analyzed (Guo et al., 2001). The initial data had indicated that COMT activity was elevated in alfalfa lines with reduced CCoAOMT expression (Table 1). Interestingly, the extractable activity of CCR2 with caffeoyl CoA as substrate was also increased, between 2.5-3.7-fold, in the CCoAOMT antisense plants compared to controls (see FIG. 11).

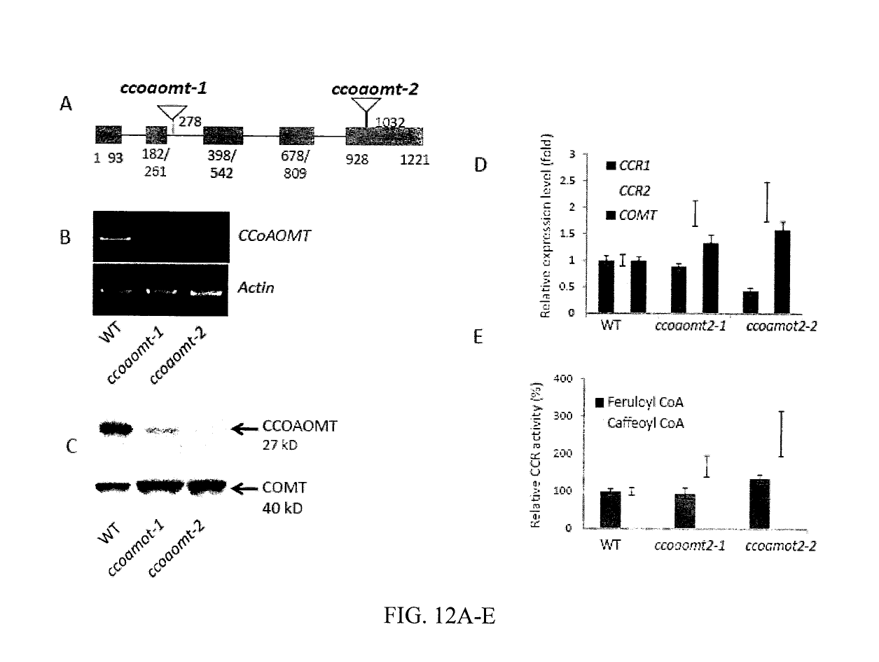

To confirm that the above findings also hold in M. truncatula, the M. truncatula Tnt1 mutant population was screened for knock-outs in CCoAOMT. Two independent lines were identified (NF5 183 and NF5347), with Tnt1 insertions at position 278 in the second intron (ccoaomt-1) and 1032 (ccoaomt-2) in the fifth exon (FIG. 12A), with strong reduction and complete loss of CCoAOMT transcripts (FIG. 12B) and immunodetectable CCoAOMT protein (FIG. 12C), respectively. Homozygous CCoAOMT Tnt1 insertion lines were of somewhat smaller stature than heterozygotes. qRT-PCR analysis indicated that CCR2 transcript levels were elevated in both CCoAOMT Tnt1 insertion lines (FIG. 12D). Extractable CCR2 activity (against caffeoyl CoA) was likewise increased in the ccoaomt mutants (FIG. 12E), but activity against feruloyl CoA was much less affected. Finally, the ccoamt mutants showed increased levels of immunodetectable COMT protein and COMT transcripts (FIG. 12C,D).

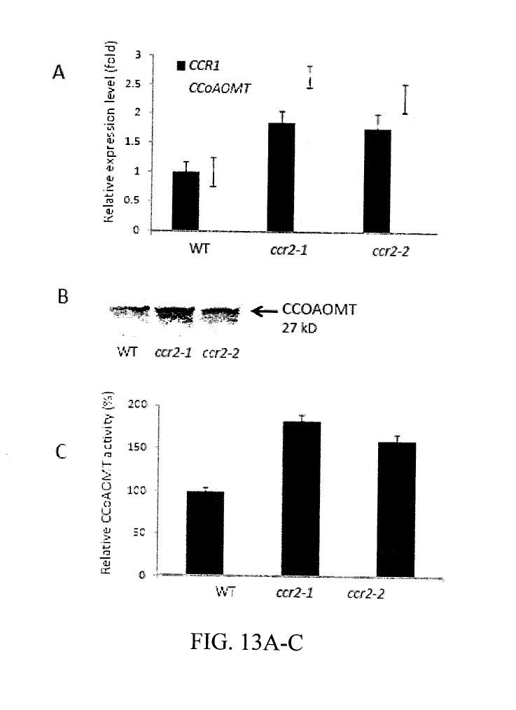

To determine whether there is a reciprocal relationship between expression of CCoAMT/CCR1 and CCR2, the three CCR2 Tnt1 transposon insertion lines already described were further analyzed. It was already demonstrated that CCR1 activity, determined with feruloyl CoA as substrate, was increased in the CCR2 mutants (FIG. 8D). qRT-PCR demonstrated that both CCR1 and CCoA OMT transcript levels were increased in the two CCR2 mutant lines (FIG. 13A), and CCoAOMT protein level (FIG. 13B) and extractable enzymatic activity (FIG. 13C) were likewise increased.

### Example 9

### Discussion of Studies

### Genetic Evidence of a Role for COMT as a Monolignol 3-O-Methyltransferase

In spite of earlier proposals suggesting that the pathway to monolignols could operate as a metabolic grid (reviewed in Dixon et al., 2001), a more linear pathway has been favored in recent years (Humphreys and Chapple, 2002). Studies on COMT and CCoAOMT single and double mutants in Arabidopsis provided the first evidence for redundancy at the level of introduction of the 3-O-methyl group of monolignols, and challenged the notion that the function of COMT was primarily the introduction of the 5-O-methyl group (Do et al., 2007). However, the phenotype of the COMT/CCoAOMT double knock-out in Arabidopsis is very severe, and it is therefore not easy to conclude whether alterations in lignin content and composition are the result of simply altering monolignol supply, or reflect dramatic developmental changes. To help address this problem a parallel approach, but using RNAi down-regulated lines was demonstrated here.

COMT/CCoAOMT double knock-down lines of alfalfa show significantly reduced growth compared to lines down-regulated in either enzyme alone, but several could be grown to maturity and their lignin contents/compositions compared at the same developmental stage as that of the controls. As observed previously, down-regulation of COMT alone resulted in a massive loss of S lignin units, whereas down-regulation of CCoAOMT alone had a much greater effect on G than on S units. Interestingly, the S lignin levels in COMT: CCoAOMT double knock-down lines was invariably higher than in the COMT knock-down lines. This may be explained by the higher COMT activity in the double knock-downs than in the single COMT knockdowns, consistent with an earlier observation (Guo et al., 2001).

The structure of the xylem elements of the double OMT knock-down lines was quite unusual, with the walls being highly thickened with an amorphous appearance. Although independent down-regulation of several of the enzymes in the monolignol pathway leads to distorted xylem element walls in alfalfa (Nakashima et al., 2008) and Arabidopsis (Patten et al., 2010), none demonstrates this appearance. The fold-reduction of thioacidolysis yield in the double OMT knock-down lines is as severe as in lines down-regulated in HCT (Chen et al., 2006), but the phenotype of the vascular elements is very different, suggesting that the unusual doughnut-shaped cell walls observed in COMT:CCoAOMT down-regulated lines are not simply the result of low lignin levels but may also reflect profound changes in polysaccharide deposition patterns.

Reduction of COMT activity in a CCoAOMT down-regulated background led to a much greater loss of G lignin than observed in the lines only down-regulated in CCoAOMT. This supports the hypothesis that COMT can act redundantly with CCoAOMT in vivo to catalyze the 3-O-methylation of a caffeoyl moiety destined for G monomer biosynthesis in plants in which the CCoAOMT step is blocked. Based on the known in vitro substrate specificity of alfalfa COMT (Parvathi et al., 2001), the substrate for this reaction is most likely caffeoyl aldehyde.

### Biochemical Properties of Medicago CCR Forms

CCR is the first committed step specific for monolignol biosynthesis. The first CCR gene was cloned and characterized from Eucalyptus (Lacombe et al., 1997), and subsequently from tobacco (Piquemal et al., 1998). In silico analysis of the Arabidopsis genome sequence databases indicated 11 annotated CCR homologs (Costa et al., 2003). However, only two isoforms, Arabidopsis CCR1 and CCR2, have been characterized at the enzymatic level and proven to be true CCR enzymes (Lauvergeat et al., 2001), and both prefer feruloyl CoA as substrate.

Substrate specificity and kinetic analyses indicate that the two functionally active Medicago CCRs possess quite different catalytic properties. CCR1, like most CCRs characterized in other plant species (Goffner et al., 1994; Lauvergeat et al., 2001; Li et al., 2005), was most active against feruloyl and sinapoyl CoAs. In contrast, these metabolites strongly inhibited CCR2 activity at concentrations above 50 μM. A similar phenomenon has recently been reported for a CCR2 from the bioenergy crop switchgrass (Escamilla-Trevino et al., 2010). Caffeoyl CoA and 4-coumaroyl CoA, poor substrates for Medicago CCR1, are potentially allosteric activators (based on positively cooperative kinetics) and favored substrates for CCR2. These properties suggest a mechanism for the fine-tuning of monolignol biosynthesis (see below).

### In Vivo Functions of Medicago CCR1 and CCR2

Down-regulation of CCR through antisense expression dramatically reduces the amount of lignin in transgenic alfalfa (Jackson et al., 2008), poplar (Leple et al., 2007) and tobacco (Piquemal et al., 1998) and knockout mutants of Arabidopsis CCR1 showed stunted growth and delayed development (Jones et al., 2001; Goujon et al., 2003; Mir Derikvand et al., 2008). Medicago CCR1 knockout mutants exhibited an even more dramatic decrease in lignin synthesis, and the growth of the plants was so impaired that most of them could not survive under normal greenhouse conditions. Protein extracts from these plants show very strongly reduced CCR activity against feruloyl CoA. The difference in the phenotypes between Arabidopsis and Medicago CCR1 knockouts can be explained by the different biochemical properties of the second CCR form in these two species. Arabidopsis CCR2 shows reasonable activity with feruloyl and sinapoyl CoAs (Lauvergeat et al., 2001). Furthermore, the expression of Arabidopsis CCR2 was increased in CCR1 knock-out plants, which partially compensated for the decrease in CCR1 expression (Mir Derikvand et al., 2008). In contrast, Medicago CCR2, although active with feruloyl and sinapoyl CoAs at low concentrations, is unable to turn these substrates over at concentrations above 50 μM, and no significant change was found in CCR2 expression level in ccr1 knockout mutants. The increased levels of feruloyl CoA predicted to result from the knock-out of Medicago CCR1 might therefore be too high to be turned over by CCR2, thus resulting in the severe inhibition of lignin biosynthesis.

CCR2 is expressed in vascular tissues in the stem during development, unlike Arabidopsis CCR2 which is primarily expressed in response to pathogen infection (Lauvergeat et al., 2001). Furthermore, knockout of CCR2 leads to a decrease in lignin content and an increase in S/G ratio, whereas overexpression of CCR2 in Arabidopsis causes increased lignin accumulation. These observations suggest that CCR2 functions, in a partially redundant manner with CCR1, in developmentally controlled lignification. The substrate specificity of CCR2 and its preferential expression in young internodes suggest that it might function in the formation of the H lignin (via coumaroyl CoA) which is laid down early in development (Fukushima and Terashima, 1990), in addition to being a central player in the alternative pathway outlined below.

### Refining the Monolignol Pathway Model for Medicago

An earlier observation that transgenic alfalfa plants with severely reduced CCoAOMT activity still make normal levels of S lignin (Guo et al., 2001) appeared inconsistent with the currently accepted scheme for monolignol biosynthesis (Bessau et al., 2007; Vanholme et al., 2008). The relative properties and expression patterns of Medicago CCR1 and CCR2 reported here provide a potential explanation for this finding. In plants with reduced CCoAOMT activity, caffeoyl CoA levels will increase. CCR2, which is present in the same cell types as CCoAOMT, shows positively cooperative kinetics with caffeoyl CoA, resulting in increasingly efficient substrate utilization as caffeoyl CoA levels increase. This leads to the formation of caffeoyl aldehyde, a preferred substrate for 3-O-methylation by Medicago COMT (Parvathi et al., 2001). Thus, according to this model, CCR2 and COMT act together to perform the 3-O-methylation of monolignol precursors in plants in which the CCoAOMT-CCR1 pathway is not functional. This is supported by the clear reciprocal cross-talk between the two pathways that apparently functions at the transcriptional level through up-regulation of CCR2 and COMT expression when CCoAMT or CCR1 are down-regulated (and through up-regulation of CCoAOMT when CCR2 is down-regulated). However, flux through the CCR2-COMT pathway only partially compensates for the loss of flux through the CCoAOMT-CCR1 pathway, since CCoAOMT down-regulation leads to a significant reduction in G lignin levels in alfalfa.

When CCoAOMT levels are normal, it is possible that there is little flux of caffeoyl CoA through the CCR2 pathway, and knock-out of CCR2 only leads to an approximately 10% reduction in total lignin levels. The low flux through CCR2 in cells expressing CCoAOMT is likely because CCR2 is expressed at a lower level than CCR1, and is substrate-inhibited by feruloyl CoA, the product of CCoAOMT, whereas CCR1, which strongly prefers feruloyl CoA as substrate, exhibits normal Michaelis-Menten kinetics with feruloyl CoA. Thus, the function of the “CCR2-COMT shunt” in plants with non-perturbed monolignol biosynthesis still requires explanation. It would appear that the steady state level of feruloyl CoA is critical for determining the direction of flux through CCoAOMT or CCR2, and feruloyl CoA levels might also be somehow involved in orchestrating the cross-talk of the two routes at the gene expression level. Feruloyl CoA is also a substrate for feruloylation of pectic and hemicellulosic polysaccharides in the cell wall (Yoshida-Shimokawa et al., 2001; Obel et al., 2003), and the CCR2 pathway might function to maintain lignin biosynthesis in cells in which feruloyl CoA is being shunted into other such pathways. That this pathway might exist in species other than Medicago is suggested by the observation that Arabidopsis ccr1 mutants exhibit a significantly smaller reduction in extractable CCR activity against caffeoyl CoA than against feruloyl CoA, pointing to the existence of an additional enzyme with preference for caffeoyl CoA. Such an enzyme has also recently been described in switchgrass (Escamilla-Trevino et al., 2010).

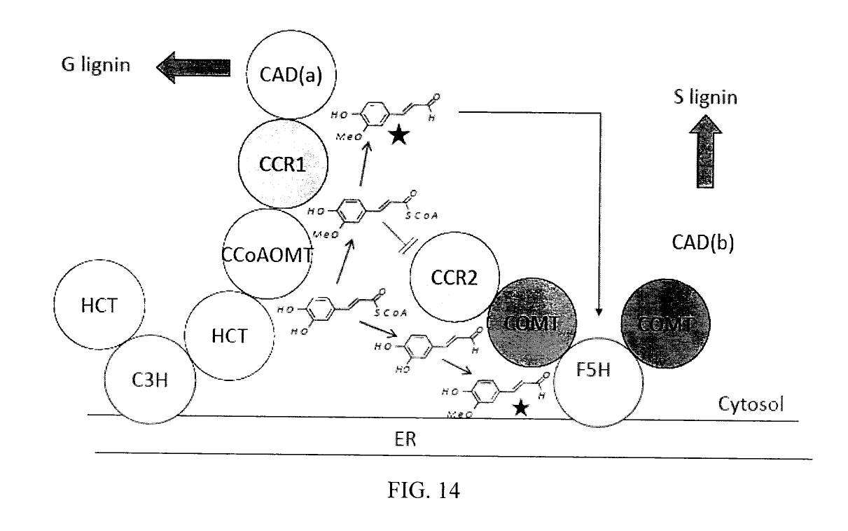

Although the existence of the CCR2-COMT shunt explains the maintenance of overall lignin biosynthesis in CCoAOMT down-regulated plants, it does not explain why S lignin is maintained at the expense of G lignin, since both reduction/methylation pathways ultimately produce coniferaldehyde. Although the substrate inhibition of CCR2 by sinapoyl CoA is even more striking than by feruloyl CoA, the currently accepted pathway to sinapyl alcohol does not involved reduction of sinapoyl CoA by a CCR enzyme. The possibility of metabolic channeling centered on membrane associated cytochrome P450 enzymes has been much discussed in relation to phenylpropanoid biosynthesis (Liu and Dixon, 2001; Ro and Douglas, 2004; Winkel, 2004). In this respect, it is possible that the “early” involvement of COMT as a 3-O-methyltransferase in the CCR2-COMT shunt places intermediates in a metabolic channel through associations of COMT with the membrane-bound ferulate/coniferaldehyde 5-hydroxylase (F5H), its potential channeling partner during its involvement in S lignin biosynthesis (FIG. 14).

In summary, a pathway for monolignol biosynthesis is described in which the methylation followed by reduction of caffeoyl CoA is bypassed by a shunt in which caffeoyl CoA is first reduced and then 3-O-methylated by COMT. This pathway was first proposed based on studies of the substrate preference of COMT (Paravthi et al., 2001) and has been subsequently included by some authors in their diagrams of the monolignol pathway (Li et al., 2008; Weng et al., 2010). The characterization of the unique properties of Medicago CCR2 now provides the first clear experimental evidence for this pathway and its possible function in vivo, indicates that the designation of COMT as a 5-hydroxyconiferaldehye O-methyltransferase that only functions in S lignin biosynthesis in vivo (Osakabe et al., 1999; Vanholme et al., 2008) was perhaps premature, opens up interesting avenues for exploring transcriptional crosstalk in monolignol biosynthesis, and potentially provides a new tool for genetic modification to increase lignin biosynthesis in plants.

### REFERENCES

The following references, to the extent that they provide exemplary procedural or other details supplementary to those set forth herein, are specifically incorporated herein by reference.
U.S. Pat. No. 4,461,648; U.S. Pat. No. 4,535,060; U.S. Pat. No. 5,000,000; U.S. Pat. No. 5,037,663; U.S. Pat. No. 5,302,523; U.S. Pat. No. 5,322,783; U.S. Pat. No. 5,384,253; U.S. Pat. No. 5,464,765; U.S. Pat. No. 5,508,184; U.S. Pat. No. 5,538,877; U.S. Pat. No. 5,538,880; U.S. Pat. No. 5,545,818; U.S. Pat. No. 5,550,318; U.S. Pat. No. 5,563,055; U.S. Pat. No. 5,591,616; U.S. Pat. No. 5,610,042.
U. S. Pat. Publ 20040049802
Abdullah et al., Biotechnology, 4:1087, 1986.
Alexander, Stain Technol., 44: 117-122, 1969.
Bates, Mol. Biotechnol., 2(2):135-145, 1994.
Battraw et al., Theor. App. Genet., 82(2):161-168, 1991.
Bevan et al., Nucleic Acids Research, 11(2):369-385, 1983.
Bhattacharjee et al., J. Plant Bioch. and Biotech. 6(2):69-73, 1997.
Bouchez et al., EMBO Journal, 8(13):4197-4204, 1989.
Bower et al., Plant Journal, 2:409-416. 1992.
Buising et al; Mol Gen Genet, 243(1):71-81. 1994.
Callis et al., Genes Dev., 1:1183-1200, 1987.
Casa et al., Proc. Natl. Acad. Sci. USA, 90(23):11212-11216, 1993.
Chandler et al., The Plant Cell, 1:1175-1183, 1989.
Chen and Dixon, Nature Biotechnology, 25: 759-761, 2007.
Christou, et al., Proc. Natl. Acad. Sci. USA, 84:3962-3966, 1987.
Chu et al., Scientia Sinica, 18:659-668, 1975.
Conkling et al., Plant Physiol., 93:1203-1211, 1990.
De Block et al., EMBO Journal, 6(9):2513-2518, 1987.
De Block et al., Plant Physiol., 91:694-701, 1989.
Dellaporta et al., Chromosome Structure and Function: Impact of New Concepts, 18th Stadler Genetics Symposium, 11:263-282, 1988.
Devane et al., Science, 258:1946-1949, 1992
D'Halluin et al., Plant Cell, 4(12):1495-1505, 1992.
Dixon, et al., Rec Adv Phytochem., 28:153-178, 1994.
Djerbi et al., Planta, 221: 739-746, 2005.
Downward, BMJ, 328(7450):1245-1248, 2004.
Duff and Murray, Bioresource Tech., 55:1-33, 1995.
Ebert et al., 84:5745-5749, Proc. Natl. Acad. Sci. USA, 1987.
Ellis et al., EMBO Journal, 6(11):3203-3208, 1987.
Fire et al., Nature, 391: 806-11, 1998.
Fraley et al., Bio/Technology, 3:629-635, 1985.
Fromm et al., Nature, 319:791-793, 1986.
Fukushima and Hatfield, J. Agric. Food Chem. 52:3713-3720, 2004.
Gallie et al., The Plant Cell, 1:301-311, 1989.
Ghosh-Biswas et al., J. Biotechnol., 32(1):1-10, 1994.
Gong et al., Adv. Biochem. Engng. Biotech. 65: 207-241, 1999.
Hagio et al., Plant Cell Rep., 10(5):260-264, 1991.
Hamilton et al., Proc. Natl. Acad. Sci. USA, 93(18):9975-9979, 1996.
Haseloff et al., Proc. Natl. Acad. Sci. USA, 94(6):2122-2127, 1997.
Hatfield et al., Crop Science, 39: 27-37, 1999.
He et al., Plant Cell Reports, 14 (2-3):192-196, 1994.
Hensgens et al., Plant Mol. Biol., 22(6):1101-1127, 1993.
Hiei et al., Plant. Mol. Biol., 35(1-2):205-218, 1997.
Hinchee et al., Bio/technol., 6:915-922, 1988.
Hou et al., Plant Physiology, 111:166, 1996.
Hu et al., Nat. Biotechnol. 17:808-812, 1999.
Hudspeth et al., Plant Mol. Biol., 12:579-589, 1989.
Ikuta et al., Bio/technol., 8:241-242, 1990.
Ishidia et al., Nat. Biotechnol., 14(6):745-750, 1996.
Jones et al., Planta, 221: 255-264, 2005.
Kaeppler et al., Plant Cell Reports, 9: 415-418, 1990.
Kaeppler et al., Theor. Appl. Genet., 84(5-6):560-566, 1992.
Katz et al., J. Gen. Microbiol., 129:2703-2714, 1983.
Keegstra and Walton, Science, 311: 1872-1873, 2006.
Kim et al., Proc Natl Acad Sci., USA 101: 1455-1460, 2004.
Klee et al., Bio-Technology, 3(7):637-642, 1985.
Knittel et al., Plant Cell Reports, 14(2-3):81-86, 1994.
Lapierre et al., Res. Chem. Intermed. 21: 397-412, 1995.
Lawton et al., Plant Mol. Biol., 9:315-324, 1987.
Lazzeri, Methods Mol. Biol., 49:95-106, 1995.
Lee et al., Adv. Biochem. Engng. Biotech., 65: 93-115, 1999
Lee et al., Korean J. Genet., 11(2):65-72, 1989.
Lehner et al., Brief Funct Genomic Proteomic, 3(1):68-83, 2004.
Lorz et al., Mol Gen Genet, 199:178-182, 1985.
Marcotte et al., Nature, 335:454, 1988.
Mes-Hartree, et al., Appl. Microbiol. Biotechnol., 29:462-468, 1988.
Mitsuda et al., Plant Cell, 17: 2993-3006, 2005.
Morjanoff and Gray, Biotechnol. Bioeng. 29:733-741, 1987.
Murakami et al., Mol. Gen. Genet., 205:42-50, 1986.
Murashige et al., Physiol. Plant, 15:473-497, 1962.
Nagatani et al., Biotech. Tech., 11(7):471-473, 1997.
Odell et al., Nature, 313:810-812, 1985.
Ogawa et al., Sci. Rep., 13:42-48, 1973.
Olsson et al.; Enzyme and Microb. Technol. 18:312-331, 1996.
Omirulleh et al., Plant Mol. Biol., 21(3):415-428, 1993.
Ow et al., Science, 234:856-859, 1986.
PCT App. WO 94/09699
PCT App. WO 95/06128
PCT App. WO 97/41228
PCT App. WO 97/4103
PCT App. WO 92/17598
Pfaffl, Nucleic Acids Res., 29: e45, 2001.
Potrykus et al., Mol. Gen. Genet., 199:183-188, 1985.
Prasher et al., Biochem. Biophys. Res. Commun., 126(3):1259-1268, 1985.
Ramakers et al., Neurosci Lett., 339: 62-66, 2003.
Reddy et al., Proc. Nat. Acad. Sci., 102 :16573-16578, 2005.
Reichel et al., Proc. Natl. Acad. Sci, 93 : 5888-5893. 1996.
Reynolds, Nat. Biotechnol. 22 :326-330, 2004.
Rhodes et al., Methods Mol. Biol., 55:121-131, 1995.
Richards et al., Plant Cell Rep. 20:48-54, 2001.
Ritala et al., Plant Mol. Biol., 24(2):317-325, 1994.
Rogers et al., Methods Enzymol., 153:253-277, 1987.
Sambrook et al., In: Molecular Cloning-A Laboratory Manual (second edition), Cold Spring Harbour Laboratory Press, 1989.
Sheen et al., Plant Journal, 8(5):777-784, 1995.
Singsit et al., Transgenic Res., 6(2):169-176, 1997.
Somleva et al., Crop Science, 42:2080-2087, 2002.
Stalker et al., Science, 242:419-422, 1988.
Sullivan et al., Mol. Gen. Genet., 215(3):431-440, 1989.
Sun and Cheng, Bioresource Technol. 83:1-11, 2002.
Sutcliffe, Proc. Natl. Acad. Sci., 75:3737-3741, 1978.
Tadege et al., Trends Plant Sci., 10: 229-235, 2005.
Tadege et al., Plant J., 54: 335-347, 2008.
Thillet et al., J. Biol. Chem., 263:12500-12508, 1988.
Thomas et al., Plant Sci. 69:189-198, 1990.
Thompson et al., Euphytica, 85(1-3):75-80, 1995.
Thompson et al., The EMBO Journal, 6(9):2519-2523, 1987.
Tian et al., Plant Cell Rep., 16:267-271, 1997.
Tingay et al., The Plant Journal, (6) p. 1369-1376. 1997.
Tomes et al., Plant. Mol. Biol. 14(2):261-268, 1990.
Torbet et al., Crop Science, 38(1):226-231, 1998.
Torbet et al., Plant Cell Reports, 14(10):635-640, 1995.
Toriyama et al., Theor Appl. Genet., 73:16, 1986.
Tsukada et al., Plant Cell Physiol., 30(4)599-604, 1989.
Twell et al., Plant Physiol., 91:1270-1274, 1989.
Uchimiya et al., Mol. Gen. Genet., 204:204, 1986.
Vasil et al., Plant Physiol., 91:1575-1579, 1989.
Walker et al., Proc. Natl. Acad. Sci. USA, 84:6624-6628, 1987.
Wang et al., Molecular and Cellular Biology, 12(8):3399-3406, 1992.
Wyman, Annu. Rev. Energy Environ. 24:189-226, 1999.
Yamada et al., Plant Cell Rep., 4:85, 1986.
Yang et al., Proc. Natl. Acad. Sci., 87:4144-4148, 1990.
Zhang et al., Science 267:240-243, 1995.
Zhong et al., Plant Cell, 20: 2763-2782, 2008.
Zhong et al., Planta, 225: 1603-1611, 2007.
Zhou et al., Plant Cell Reports, 12(11).612-616, 1993.
Zukowsky et al., Proc. Natl. Acad. Sci. USA, 80:1101-1105, 1983.

What is claimed is:

1. A nucleic acid molecule selected from the group consisting of:
(a) a nucleic acid sequence that hybridizes to SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70, under conditions of 1×SSC, and 65° C. and encodes a polypeptide with caffeoyl CoA reductase activity;
(b) a nucleic acid sequence encoding a polypeptide with at least 85% amino acid identity to SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71, having caffeoyl CoA reductase activity;
(c) a nucleic acid sequence with at least 85% identity to SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70 and encodes a polypeptide with caffeoyl CoA reductase activity; and
(d) the complement of a sequence of (a)-(c)
wherein the nucleic acid sequence is operably linked to a heterologous promoter.

2. A recombinant vector comprising the nucleic acid molecule of claim 1.

3. The recombinant vector of claim 2, wherein the promoter is a plant developmentally-regulated, organelle-specific, inducible, tissue-specific, constitutive, or cell-specific promoter.

4. The recombinant vector of claim 2, defined as an expression cassette.

5. An isolated polypeptide having at least 85% amino acid identity to the amino acid sequence of SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 or SEQ ID NO:71, or a fragment thereof having caffeoyl CoA reductase activity.

6. A transgenic plant transformed with the nucleic acid molecule of claim 1.

7. The transgenic plant of claim 6, wherein the plant is a switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), soybean, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass, tomato, or poplar plant.

8. The transgenic plant of claim 6, wherein the plant exhibits increased lignin content in selected tissues relative to those tissues in a second plant that differs from the transgenic plant only in that the nucleic acid molecule is absent.

9. The transgenic plant of claim 6, further defined as a fertile R₀ transgenic plant.

10. The transgenic plant of claim 6, further defined as a progeny plant of any generation of a fertile R₀ transgenic plant, wherein the transgenic plant comprises the nucleic acid molecule of claim 1.

11. A plant part comprising the nucleic acid molecule of claim 1.

12. A seed comprising the nucleic acid molecule of claim 1.

13. A cell transformed with the nucleic acid sequence comprises a nucleic acid molecule of claim 1.

14. A method of increasing lignin content in a plant, comprising expressing in the plant the nucleic acid molecule of claim 1.

15. The method of claim 14, wherein the nucleic acid sequence has been introduced into the plant by plant breeding.

16. The method of claim 14, wherein the nucleic acid sequence has been introduced into the plant by genetic transformation of the plant.

17. The method of claim 14, wherein the heterologous promoter is a constitutive or tissue specific promoter.

18. The method of claim 14, wherein the plant is a switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), soybean, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass, tomato or poplar plant.

19. The method of claim 14, further comprising preparing a transgenic progeny plant of any generation of the plant, wherein the progeny plant comprises the nucleic acid sequence.

20. A method of preparing a transgenic plant comprising transforming a plant cell with a nucleic acid molecule of claim 1 and regenerating a plant therefrom.

21. A plant part prepared by the method of claim 14.

22. A method of making a commodity product comprising:
(a) obtaining the plant of claim 6;
(b) growing the plant under plant growth conditions to produce plant tissue from the plant; and
(c) preparing a commodity product from the plant tissue.

23. The method of claim 22, wherein preparing the commodity product comprises harvesting the plant tissue.

24. The method of claim 22, wherein the commodity product is paper, paper pulp, ethanol, butanol, biodiesel, biogas, silage, carbon fiber, animal feed or fermentable biofuel feedstock.

25. A plant comprising a down-regulated CCR2 gene wherein the plant exhibits reduced lignin content.

26. The plant of claim 25, wherein the plant comprises a mutated genomic CCR2 gene.

27. The plant of claim 26, wherein the plant comprises a DNA molecule capable of expressing a nucleic acid sequence complementary to all or a portion of a CCR2 mRNA.

28. The plant of claim 27, wherein the DNA molecule comprises a nucleic acid sequence complementary to all or a portion of a CCR2 mRNA operably linked to a promoter sequence selected from the group consisting of a developmentally-regulated, organelle-specific, inducible, tissue-specific, constitutive, cell-specific, seed specific, or germination-specific promoter.

29. The plant of claim 27, wherein the nucleic acid sequence complementary to all or a portion of a CCR2 mRNA comprises a sequence complimentary to all or a portion of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70.

30. The plant of claim 26, wherein the mutated genomic CCR2 gene comprises a deletion, a point mutation or an insertion in a wild-type CCR2 gene.

31. The plant of claim 26, wherein the mutated genomic CCR2 gene is produced by irradiation, T-DNA insertion, transposon insertion or chemical mutagenesis.

32. The plant of claim 25, wherein the plant is forage plant, a biofuel crop, a cereal crop or an industrial plant.

33. The plant of claim 25, wherein the plant is a switchgrass (Panicum virgatum), giant reed (Arundo donax), reed canarygrass (Phalaris arundinacea), Miscanthus33 giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), soybean, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or poplar plant.

34. The plant of claim 25, further defined as an R0 transgenic plant.

35. The plant of claim 25, further defined as a progeny plant of any generation of an R0 transgenic plant, wherein the transgenic plant has inherited the selected DNA from the R0 transgenic plant.

36. The plant of claim 25, further comprising a second DNA sequence that down-regulates lignin biosynthesis.

37. The plant of claim 36, wherein the second DNA sequence down-regulates a lignin biosynthesis gene selected from the group consisting of 4-coumarate 3-hydroxylase (C3H), phenylalanine ammonia-lyase (PAL), cinnamate 4-hydroxylase (C4H), hydroxycinnamoyl transferase (HCT), caffeic acid O-methyltransferase (COMT), caffeoyl CoA 3-O-methyltransferase (CCoAOMT), ferulate 5-hydroxylase (F5H), cinnamyl alcohol dehydrogenase (CAD), cinnamoyl CoA-reductase 1 (CCR1), 4-coumarate-CoA ligase (4CL), monolignol-lignin-specific glycosyltransferase, and aldehyde dehydrogenase (ALDH).

38. A plant part of the plant of claim 25.

39. The plant part of claim 38, further defined a protoplast, cell, meristem, root, pistil, anther, flower, seed, embryo, stalk or petiole.

40. The nucleic acid molecule of claim 1, wherein the nucleic acid sequence is complementary to all or a portion of SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68 or SEQ ID NO:70 and wherein expression of the nucleic acid molecule in a plant cell reduces lignin content of said plant cell.

41. A biofuel feedstock comprising a nucleic acid molecule of claim 40.

42. A method of increasing the level or availability of one or more fermentable carbohydrates in a biofuel crop species plant comprising down-regulating CCR2 gene expression in the plant.

43. A method of decreasing the lignin content in a plant comprising down-regulating CCR2 gene expression in the plant.

44. A method for increasing the digestibility of a forage crop comprising down-regulating CCR2 gene expression in the plant.
